(19) United States
(12) Patent Application Publication
(10) Pub. No.: US 2025/0354199 A1
(43) Pub. Date: Nov. 20, 2025

(54) MULTIPLEX DETECTION WITH UNIVERSAL PROBES

(71) Applicant: Bio-Rad Laboratories, Inc., Hercules, CA (US)

(72) Inventors: George Karlin-Neumann, Hercules, CA (US); Ronald Lebofsky, Hercules, CA (US); Adam McCoy, Hercules, CA (US); Adam Bagnall Conn, Pinole, CA (US)

(21) Appl. No.: 19/208,750

(22) Filed: May 15, 2025

Related U.S. Application Data
(60) Provisional application No. 63/648,486, filed on May 16, 2024.

(51) Int. Cl. C12Q 1/6816; C12Q 1/6876

(52) U.S. Cl. CPC C12Q 1/6816; C12Q 1/6876

(57) ABSTRACT

The invention provides methods for the detection of molecular targets by digital PCR (dPCR) using a set of universal probes and target-specific tailed primers. Each target is amplified by a unique mixture of primers. The tailed amplicons anneal to a universal set of probes to detect the associated targets. Some targets are amplified using more than one tailed primer. Some targets are amplified using the same multiple tailed primers. In these embodiments, the primers are concentrated to produce a different number of amplicons for each tailed primer, resulting in a different probe-amplicon balance for each target. Two colors of fluorescence intensity are read and plotted as a 2D plot. In the plot, different targets contribute well-resolved clusters. Each cluster in the plot essentially lies a long its own radius allowing for radial multiplexing. The use of a universal set of probes in multiple assays provides for greater flexibility and throughput.

### TECHNICAL FIELD

The disclosure relates to digital PCR with universal oligonucleotide probes for the simultaneous detection of multiple targets.

### BACKGROUND

Numerous clinical and research methods involve the detection of specific nucleic acids. For example, cancer is typically associated with certain mutations in tumor DNA. Accordingly, the ability to detect those mutations can be informative of the presence or progress of a cancer or indicative of the success of a treatment. In theory, after a patient is treated to remove a tumor, the progress of the cancer can be evaluated later by performing an assay on a blood sample to detect circulating tumor DNA (ctDNA). Detecting these tumor nucleic acids in a laboratory assay would be a valuable diagnostic tool.

Unfortunately, circulating tumor DNA may contain myriad variants of interest. It would be time consuming to perform next generation sequencing (NGS) on ctDNA and analyze the resultant reads to report all variants present. Similar challenges arise in pathogen detection, agriculture, gene expression, proteomics, and other fields of endeavor. For example, there is increasing interest in assaying wastewater to monitor patterns of viral spread in communities. However, in such a sample, there may be great genetic diversity among those viral nucleic acids that are present. For example, a wastewater system that serves a large metropolitan region may have trace amounts of viral nucleic acid that are variously derived from different variants of a virus that is spreading through the region. Even with the ability to capture those variants by polymerase chain reaction (PCR), it may be costly and time-consuming to run multiple assays for capture and detection of the different variants of interest.

### SUMMARY

The invention provides methods for improved multiplexed detection of target nucleic acids by digital PCR (dPCR) using universal probes. For the multiplex dPCR of the invention, a sample is divided into a plurality of aqueous partitions with PCR reagents and each partition is subject to conditions that promote amplification by PCR. The provided reagents include, for each target of interest, a primer set in which at least one primer has a universal tail. The reagents also include universal probes-probes that are not designed to be complementary to genomic sequences of any organism of interest but instead designed to be complementary to the universal tails provided on the primers or their reverse complements. For each target of interest, a set of primers unique to that target is provided, which set may include a single primer pair with one universal tail or a combination of primer pairs that include a mixture of, e.g., two different universal tails (to be amplified in the presence of at least two different fluorescent probes). Notably, primer pairs of the invention are designed against target loci of interest and may simply be DNA oligonucleotides, while probes of the invention are “universal” in that they are specifically designed to anneal to probe-binding sites and the probes are preferably fluorescently-labeled oligos that may include fluorescent quenchers. Due to each target having its own set of primers and/or their reverse complements that will anneal to a combination of probes specific to the primers in a probe combination that is unique to that target, each target will produce a combination of fluorescent color and intensity (from the probes) that is specific to that target. The synthesis of the reverse complements of the universal primer tails may occur during the amplification reaction in the partitions.

The probe and primer combinations may be used to detect multiple, e.g., more than two, targets independently even with detection instruments that only detect, or “read”, two color signals at a time, i.e., two-channel detection instruments. For example, where four targets are to be detected in a two-channel instrument: a first target may be amplified with one primer pair having a first universal tail whose reverse complement is bound by a first fluorescent probe; a second and third target may be amplified by two primer pairs (e.g., in unequal concentrations) having the reverse complement of the first and second universal tails to which the first probe and a second probe bind but where the second target is probed with a higher quantity of the first universal tail while the third target is probed a higher quantity of the second universal tail; and a fourth target may be probed entirely by hybridizing to the reverse complement of the second universal tail. In that example if the first and second probes carry fluorophores of color 1 and color 2, respectively, partitions carrying the first target will give a signal of all color 1; partitions carrying the second target will give a high amount of color 1 and a minor amount of color 2; partitions with the third target will give minor color 1 and abundant color 2; and partitions carrying target four will give a fluorescent signal of all color 2. From that logic, it can be understood that four distinct targets can be detected independently (“multiplexed”) while reading partitions using a two-channel instrument.

The multiplexing may be expanded. For example, by varying the primer ratios, more than four (e.g., 5 or 6 or more) targets may be detected in a two-channel instrument. Also, separately, multiple targets may be read in an instrument that reads a higher number of channels. For example, instruments referred to as 6-channel digital PCR instruments may be used to read any arbitrary number of targets (e.g., 2, 4, 5, 7, 12, 15, 16, 20, 50, etc.) in a single instrument run. Each partition will have a fluorescent signal specific to a target that is in that partition in the color channels for which primers and probes are specific to that target.

Approaches to multiplexing employed in the disclosure generally include radial multiplexing, intensity multiplexing, others, or combinations thereof. Radial multiplexing generally refers to the representation of each partition as a point in a two-dimensional space. In such approaches, each partition will generate one point to the space. Radial multiplexing may involve depicting those points on a 2D graph or plot, on which each target that is present in the sample will appear as a distinct cluster of points in a defined angular position with respect to the plotted color axes. With knowledge of the primer pairs and probes that were used to interrogate the sample, such a plot may be analyzed (e.g., by software) to detect the clusters and quantify the associated targets in the sample. A similar approach, generally referred to herein as computational multiplexing, provides a similar result without the need to generate any 2D graph or plot. For computational multiplexing, fluorescent signal is read from each partition and stored or delivered to an analysis package (e.g., software) in for example a tabulated or data normal format. The analysis package can assign each partition to a location in a 2D or 3D space (without necessarily displaying anything in a human-readable form). The analysis package can detect the clusters and “call” each as to which target it represents in the sample. Intensity multiplexing is distinct, albeit with some similarities, and involves providing each target with a unique quantity of primers and/or probes such that each target in a partition imbues that partition with a level of fluorescent intensity from that partition specific to the target contained therein.

An important feature of the invention is the use of universal probes. More specifically, the universal probes comprise a specific set of universal probes combined at a fixed concentration. As used herein, “universal” means that a probe is designed to anneal to a cognate synthetic probe binding sequence and that neither the probe sequence nor the complementary probe binding sequence are designed to anneal to any site in a genome of interest. In fact, it may be preferable to design probes (and binding sites) without matches in any genome of interest such as human, mouse, corn, soybean, etc. In preferred embodiments, targets of interest are amplified using a target-specific primer pair in which at least one primer has a universal probe binding sequence, or a reverse complement thereof, in a 5′ tail of the primer (a “universal tail”). In other embodiments, the universal probe binding sequence may be located in another portion of the primer that does not substantively complement the target sequence. A cognate universal probe is provided that gives a detectable signal when the target is amplified using the target-specific primer pair. A plurality of distinct universal probes may be used such as, for example, 2 or 6 or any other number. Each universal probe is linked to a fluorophore or dye, preferably of a color that is read by the channel of an optical instrument such as a 2-channel or 6-channel digital PCR (dPCR) instrument. The universal probes may contain dye quenchers. The universal probes are preferably not target specific. As a consequence, the probes may be made independently of any particular biological assay. In fact, sets of probes may be synthesized and held on-hand as a reagent for a dPCR assay that is performed later. Then, when there is call for a digital PCR assay to detect a set of specific targets, one needs only to have appropriate primer pairs synthesized for use in a dPCR instrument with the pre-existing universal probe as a reagent. This is beneficial because it is comparatively easier, faster, and less expensive to order and synthesize simple DNA oligos such as primer pairs that do not contain any modifications, such as fluorophores or quenchers, than to synthesize new fluorescent probes. This is particularly useful in combination with the embodiments described herein to extend the number of detected targets beyond the number of detection channels which would otherwise require designing and manufacturing multiple probes per detection channel which is both expensive and slow. In other embodiments, the probes may be mixed to similarly achieve multiplexing higher than the number of channels.

Accordingly, the invention provides a method to achieve numerous combinations of color probes for the multiplexed detection of an arbitrary number of targets using a limited number of pre-designed universal probes (with a limited number of dyes or colors), provided as a single probe blend. For example, for a six-color instrument, only six color probes are required from a single probe blend. As another example, for a four-color instrument, only four probes are required from a single probe blend. In summary, for an X-color instrument, X color probes are required from a single probe blend. As will be apparent to the skilled artisan, the ability to provide the probes from the same color probe blend for multiple different assays greatly simplifies the inventory and workflow issues identified above, as well as other issues common to designing assay sets.

Although not required, it is also possible for more colors of probes to be used with fewer colors of analysis. For example, six color probes may be used with a 6-color instrument or a 4-color instrument.

These probes may be configured in different ways to facilitate multiple assay options. In one such example, the 6 probes may be composed of 4 total colors where 2 probes are labeled with the same color for two of the colors, for example to provide easier amplitude modulation in the signals of those channels or to provide multiple options such that primers that have a sequence issue with one tail could still be designed with a separate tail to use the preferred detection channel. In another example, the 6 probes may be labeled with 6 colors, two of which are not used in that particular 4 color instrument but may be used in other instruments. In still another example, the 6 probes may be labeled with 6 colors, 4 of which the instrument is calibrated for and two of which result in signals the instrument interprets as mixtures of its 4 color channels.

In certain aspects, the invention provides methods of detecting nucleic acid targets. The methods comprise partitioning a sample comprising at least two targets into a plurality of partitions. The methods may also be applied to more numerous targets (e.g., more than two, three or more, four or more, five or more, six or more, more than six, etc.). The partitions are formed to contain amplification reagents, a set of universal probes, and primers with universal tails, each primer at a concentration specific to each target. The set of universal probes (e.g., fluorescent hydrolysis probes) are not specific to any one of targets, but rather universal to the tailed primers that anneal to the targets during amplification. The set of universal probes may also be specific to the reverse complement of the tailed primers that are synthesized as a product of PCR. In this way, the specificity of the fluorescence reaction lies in the reaction of universal probes with specific primers, rather than the use of specific probes for each target.

The probes, after reaction with the tailed primers or reverse complement thereof, produce fluorescence of a respective color. Notably, in some embodiments, at least some of the tailed amplicons are targeted by a mixture of probes with two different colors of fluorescent reporter. To illustrate, a first target may be targeted by a universal probe with a carboxyfluorescein reporter (FAM), a third target may be targeted by a universal probe with a hexachlorofluorescein reporter (HEX), while a second target may be targeted by both of the probes, some with FAM, some with HEX. More complex multiplexing schemes using more robust probe mixtures are described herein.

The partitions are subject to conditions that promote amplification (e.g., thermocycling) and the methods include reporting the presence or absence of the targets in the sample based on the amounts of the colors from the set of probes detected from the partitions. In some embodiments, four targets are detected and the fourth of the targets may be a wild-type while the other three targets are mutant versions of the wild-type. In some embodiments, four targets are detected, and the targets comprise four different RNA transcript species. The methods provide a droplet digital PCR (ddPCR) assay with at least a two-color readout in which two or more than two targets can each be independently detected in a sample.

In support of readouts such as ddPCR, the methods involve portioning the sample and reagents into partitions. The partitions may be droplets, wells in a plate, or other fluid portioning structure. The partitions may be monodisperse or heterodisperse, i.e., vary in size. The partitions may be characterized by having different, yet pre-defined sizes. Methods preferably include diluting the sample so that each of the partitions receives a limited number of target molecules, such as zero, one, two, sometimes three, and a very small number of four or more. Methods may include diluting the sample such that a proportion of partitions contain no targets. The dilution can be calculated so that a majority of partitions receive a target number (e.g., zero or one) of targets. Each target molecule will serve as a template for generation of amplicons using specific tailed primers in the presence of universal fluorescence probes.

Preferred primers for amplification have a tail which reacts with one of the probes after amplification. The amplicons produced by the amplification reaction include the complement of the primer tail and provide the substrate for binding of the universal probe. Stated differently, the target is amplified using a tailed forward primer and a reverse primer. Another embodiment may involve using tailed forward and tailed reverse primers. Yet another embodiment may involve using a combination of tailed and un-tailed forward primers, reverse primers and combinations thereof. A set of primers is provided to each partition. Accordingly, in some embodiments, the primers are provided at concentrations specific to the targets to be detected.

Preferred probes include an oligonucleotide backbone that anneals in a sequence specific manner to a substrate of that probe, plus a fluorophore and a quencher. Methods may include thermocycling the droplets within a reaction tube or well of a plate. During amplification with polymerase, exonuclease activity of the fluorophore digests the oligonucleotide backbone of any bound probe, separating the fluorophore from the quencher, allowing the fluorophore to fluoresce during a readout step. To read fluorescence, methods may include flowing the droplets (e.g., one-at-a-time) past a detector (and optionally a light source for excitation of fluorophores).

The amplification step may include thermocycling, using reagents that include PCR primers and dNTPs. Preferably, the probes include fluorescent probes such as molecular beacons that each anneal to a sequence of one of the tailed amplicons. Molecular beacon probes may include an oligonucleotide loop backbone that anneals in a sequence specific manner to a substrate of that probe, complementary stem sequences, plus a fluorophore and a quencher. During the amplification reaction, the complement to the loop sequence is synthesized followed by hybridization of the loop sequence, separating the fluorophore and quencher, allowing the fluorophore to fluoresce during the readout step. During molecular beacon detection reactions, the amplification reaction may use polymerases that have exonuclease activity or polymerases that lack exonuclease activity.

Methods may be used for multiplex detection of multiple targets with two-channel readout at a time. For example, five targets may be read in two color channels. In some embodiments, four or six or more color channels are used. For example, the method may include reading the sample for at least seven variants using at least six colors, wherein the detecting step reads two of the six colors, in two channels, at a time. The six colors may be provided by any suitable fluorophores or fluorescent dyes. Some embodiments use six fluorescent reporters that include carboxyfluorescein (FAM), hexachlorofluorescein (HEX), cyanine5 (CY5), cyanine5.5 (CY5.5), carboxy-rhodamine (ROX), and a fluorescent oligonucleotide dye with 594 nm adsorption (ATTO590). Systems and methods of the invention are well suited for the detection of one or two targets simultaneously in a two-channel instrument or by simultaneous reading of a multichannel dPCR instrument. Even if an assay need only detect one or two targets in two color channels, methods disclosed herein may be preferable to conventional methods due to the use of the disclosed universal probes and related methods of use. A user may simply have sets of universal probes on-hand, e.g., as a reagent. On deciding to test a sample for one or two targets, the user need only order primer pairs with 5′ universal tails, i.e., simple DNA oligos, to run the assay. These tailed primers may not contain fluorophores and/or quenchers.

Methods may be used for radial multiplexing, which can be implemented by reading two colors from each of a plurality of partitions, plotting intensity of the two colors on a 2D or 3D plot with axes for intensity of each color and identifying clusters of points on the plot. Each cluster will typically be found essentially lying along distinct radius extending from a cluster corresponding to double-negative partitions (no significant fluorescence of either color).

In radial multiplexing, clusters corresponding to distinct targets in the sample are distinguishable according to their different radial directions from the double negative cluster. Clusters can further be distinguished based on radial distance. Thus, five or more targets can be distinguished by providing mixtures of primers with varying amounts of primers specific to each target and a universal probe set interrogated in the two channels. Other methods of multiplexing such as fluorescence intensity multiplexing are within the scope of the disclosure. Radial multiplexing may also be performed by a software package that does not create a 2D plot but that performs a function such as tabulating fluorescence detection from each partition, assigning each partition to a location in a 2D space, detecting clusters among the assigned locations, and reporting the presence of targets based on the clusters.

While embodiments of radial multiplexing are described above with respect to reading and plotting two colors (i.e., two color channels), it is also envisioned that primers for a single target may be engineered to be detected in more than two color channels. For example, a target may be detected in three channels, four channels, five channels, six channels, or more than six channels.

For radial multiplexing, the reporting step may include plotting the amounts of a first color and a second color detected from the partitions as points on a graph, e.g., 2D plot, and identifying clusters of the points on the graph corresponding to the presence of any of the targets. The quantities of the respective targets in the sample may be determined by a Poisson model of templates into the partitions that would generate the observed pattern of clusters.

The reporting step may include detecting colors from the partitions, two color channels at a time, in three detection operations for six colors. The reporting step may include detecting colors from partitions, six color channels at a time in a single detection operation for six colors. The reporting step may include reporting the presence or absence of at least seven targets being present in the sample.

In some embodiments, the method is used to detect mutant versions of, or variants of, a gene. To give but one illustrative embodiment, the gene may be estrogen receptor 1 (ESR1) and one or more of the seven mutations may be selected from the group consisting of c1138G>C, c.1387T>C, c1607T>G, c.1609T>A, c1610A>C, c1610A>G, and c.1613A>G in the coding sequence of the ESR1 gene. In another example, the targets are present in tumor DNA in the sample (e.g., which has previously been sequenced to detect tumor-specific variants), and the method includes (i) isolating circulating tumor DNA (ctDNA) from the sample, or (ii) isolating circulating tumor cells (CTCs) from the sample and purifying tumor DNA from the CTCs; such tumor DNA may be analyzed to detect the tumor-specific variants in multiplex by dPCR using universal probes according to the disclosed methods.

Preferably, each of the targets is amplified with at least one tailed primer to make tailed amplicons and the tailed amplicons are targeted with a probe or probe combination that includes at least one sequence specific to the tailed amplicons to give, as a signal, a characteristic amount of a first color and/or second color.

As opposed to previous technologies, which crafted specific probes to specific targets, the present invention utilizes universal probes with specific primers for each target. In some embodiments, a first primer combination specific to a first target produces amplicons that react with a first probe to produce a first color and a second probe to produce a second color. A second primer combination specific to a second target includes a subset of primers that produce amplicons that react with the first probe to produce the first color and a second probe to produce a second color. Additionally, a third primer combination specific to a third target includes a subset of primers that produce amplicons that react with the first probe to produce the first color and a second probe to produce the second color, but do so with a different relative balance between the first color and the second color (based on the relative quantity of the respective amplicons produced).

Optionally, amounts of the first color and the second color detected from the partitions are plotted against respective first and second axes on a plot, wherein the three targets each form a respective distinct cluster on the plot, wherein the distinct clusters can be separated by radii extending from one point.

As opposed to previous technologies, the probes are not specific to sequences in genomes of the respective targets. Rather, the amplification primers are crafted to contain a tail which itself or the reverse complement thereof reacts with a probe, and a target-sequence specific binding region(s) to enable amplification. In so doing, the probe mixture can contain “universal” probes which are crafted to react with any amplicon engineered to contain the appropriate binding site encoded by the tail. This shift (from engineered probes to engineered primers) reduces costs and throughput time as short primers are much less expensive and easier to engineer than bulkier, more complex, luminescent probes that may require purification.

In one embodiment, four targets may be detected using six probes, each probe present at the same concentration. In this example, a first target is amplified with a set of primers which provides first amplicons; the first amplicons react with a first probe to produce a first color and a second probe to produce a second color; these reactions occur in relatively equal number. A second target is amplified, with the same set of primers, to provide a second amplicon; the second amplicons react with a third probe to produce a third color and a fourth probe to produce a fourth color; these reactions occur in unequal quantities (e.g., 25% third, 75% fourth). A third target is amplified, with the same set of primers, to provide third amplicons. As above, the third amplicons react with the third probe to produce the third color and the fourth probe to produce the fourth color; these reactions occur in unequal quantities (e.g., 75% third, 25% fourth). Finally, a fourth target is amplified with a set of primers which provides fifth amplicons; the fifth amplicons react with the first probe to produce the first color and a fourth probe to produce a fourth color in relatively equal number.

In one example of this embodiment, four targets may be detected using six probes, each probe present at the same concentration. In this example, a first target is amplified with a set of primers which provides first amplicons; the first amplicons react with a first probe to produce a first color and a second probe to produce a second color; these reactions occur in relatively equal number. A second target is amplified with a set of primers, to provide a second amplicon; the second amplicons react with a third probe to produce a third color and a fourth probe to produce a fourth color; these reactions occur in unequal quantities (e.g., 25% third, 75% fourth). A third target is amplified, with a set of primers, to provide third amplicons. As above, the third amplicons react with the third probe to produce the third color and the fourth probe to produce the fourth color; these reactions occur in unequal quantities (e.g., 75% third, 25% fourth). Finally, a fourth target is amplified with a set of primers which provides fourth amplicons; the fourth amplicons react with the first probe to produce the first color and a fourth probe to produce a fourth color in relatively equal number.

The inventors recognize that the exemplary detection method described above can be used to detect many more targets if desired. Further, some embodiments of the invention, as described above, use multiple primers corresponding to multiple probes for a single target. While the example above identifies that two primers may be used in differing quantities, some embodiments of the invention may utilize two or more primers in mixture to detect a target. For example, the target may be amplified with a set of primers including two tail sequences, three tail sequences, four tail sequences, five tail sequences, or six tail sequences. The number of tail sequences corresponds to the number of probes capable of binding to the amplicon tails. Accordingly, if more than 6 colors are analyzed, the set of primers may include more than six tail sequences, each reactive to a separate probe.

The method may include estimating quantities of each of the targets in the sample by modelling, using a computer system, a Poisson distribution of the targets that would give the reading of the signal in the two or more optical channels.

### BRIEF DESCRIPTION OF THE DRAWINGS

FIG. 1 diagrams steps of a method of the invention.

FIG. 2 illustrates a set of universal probes.

FIG. 3A shows probe primer combinations that may be used for 4 targets, A-D.

FIG. 3B shows the detection of targets A-D by radial multiplexing.

FIG. 4A shows probe primer combinations that may be used for Targets 1-7.

FIG. 4B shows the multiplexed detection of Targets 1-7.

FIGS. 5A-5B show a method using mixtures of tailed and untailed primers.

FIGS. 6A-6C show results of digital PCR on three targets using various concentrations of tailed and untailed forward primers.

FIG. 7 shows the average amplitude and cluster tightness increases across three test designs for three genetic targets for a combination (300 nM/300 nM) of tailed/untailed forward primers compared to 100 nM of forward tailed primer.

### DETAILED DESCRIPTION

Multiplexed detection of nucleic acid targets of interest using universal probes includes obtaining a sample suspected to contain the targets of interest. Methods include providing universal probes, e.g., 2 or more-sometimes 6-probes in which each probe comprises an oligonucleotide with a “universal” binding sequence linked to a fluorophore specific for that probe. The sample is divided into partitions, sometimes referred to as compartments or capsules, which may be embodied as droplets, wells (e.g., of a plate), fluidic harbors, slugs in a channel, or similar. The partitions may be monodisperse or heterodisperse, i.e., vary in size. In some embodiments, the partitions may be characterized by having different, yet pre-defined sizes. A PCR reaction is conducted in each partition with target-specific tailed primers to generate tailed amplicons. Universal probes in each partition hybridize to the reverse complement of the tailed primer in the amplicon giving a detectable signal when a cognate tailed amplicon is generated. Any number of partitions may be used (e.g., thousands). Each partition may include primers specific to several different targets (e.g., three, four, five, six, seven, or more) and fluorescent probes by which methods of the invention provide for detecting each of the targets even when using fewer detection channels than there are targets. Certain embodiments use molecular beacon probes or fluorescent hydrolysis probes that are read in two optical channels at a time.

Each target is detected with a mixture of probes that fluoresce in one or both of the two channels. One color is read from each channel (using optical detectors such as photodiodes optionally as part of a bench-top dPCR instrument) and the fluorescence intensity may be stored as data or a 2D plot with one axis for each color.

If shown as a 2D plot, each partition will appear as one distinct cluster of points on the plot based on target(s) therein, including a cluster for partitions with no targets. By careful probe design, individual clusters are well-resolved and software may be used to model a Poisson distribution of targets into partitions and report what quantity of each target in the original sample generates the observed clusters. The readout is thus multiplexed by virtue of the independent detection of multiple targets from one two-color detection operation which analyzes the combined color intensity from those two colors.

As identified above, in conventional dPCR, the design, synthesis, and purification of probes for each target is a time intensive and therefore costly process. Here, primers have universal binding sites, such as in their 5′ tails, and sequences of the universal binding sites do not substantially match nucleic acid sequences from the samples of interest. According to this disclosure, fluorescent probes have substantial sequence similarity to the universal binding sites in the primers of the invention. Under favorable hybridization conditions, the fluorescent probes hybridize to the reverse complement of the universal binding site in the tailed primers synthesized during amplicon PCR. With this design, any suitable universal sequences may be used regardless of the primer sequence, and the same fluorescently labeled probes can be used regardless of the assay and respective primer sequences. This provides cost savings and decreased turnaround time for new assays as the probes can be ordered at scale (even before an assay is performed) and new assays can be deployed without having the need to order and/or synthesize new fluorescent probes. In this regard, only unmodified and desalted tailed target specific oligos are required for the multiplex PCR reaction.

In fact, universal probe embodiments of the invention benefit end-users because, for example, a user may purchase or obtain a bench-top dPCR instrument, such as the dPCR instrument sold under the trademark QX600 by Bio-Rad Laboratories, Inc. (Hercules, CA) and then also purchase universal probes as a consumable. When the user wants to perform a new assay, there is no need to design, order, and purchase customized fluorescent probes for the new assay. The user need only obtain the relevant PCR primers, which can be simply DNA oligos obtained through the web-based ordering interface of a custom DNA provider such as Integrated DNA Technologies, inc.

Here, universal 5′ tailed sequences on primers can alleviate cost and expedite new assay deployment. A user may have a library of fluorescently labeled universal probes for each universal tailed sequence. Additionally, this disclosure provides methods of multiplexed readout (e.g., independent detection of any arbitrary number of targets using a limited number such as 2 of color channels) including, for example, radial multiplexing. In methods of the invention, radial multiplexing uses detectably-labelled probes to generate signals in a color space that is a combination of two colors. See U.S. Pat. No. 9,222,128, incorporated by reference.

The invention provides methods for detecting targets of interest and in particular provides for the use of digital PCR based detection for the multiplex detection of multiple genetic targets in a single readout operation. Some embodiments are useful for the detection of two or more, distinct genetic targets in a readout operation that employs only two color channels at a time. Other embodiments employ four, or six, or any arbitrary number of color channels at a time. Additionally, methods of the invention make use of a set of universal probes that bind to amplicons formed using target-specific primers. Accordingly, the same probe set may be used for multiple analyses regardless of the target(s) to be detected or quantified.

In preferred embodiments, the digital PCR assay may involve amplification of target nucleic acids from a sample in aqueous partitions with a primer pair including at least one tailed primer. The amplicons may be detected by a fluorescent or luminescent probe which anneals to the tailed amplicon. In the disclosed methods a set of “universal” probes is used to detect the targets. The mixture of universal probes may be used across multiple analyses to detect different targets. The specificity of detection arises from the tailed amplification primers. These primers can be engineered to selectively react with a target of interest while also including a tail engineered to react with “universal” probe. The reaction between the primer and the target of interest can be through hybridization of complementary sequences or substantially complementary sequences. Accordingly, amplification of any target with a tailed primer reactive to a probe from the set of “universal” probes can proceed without the need to engineer and manufacture a probe specific to the target of interest.

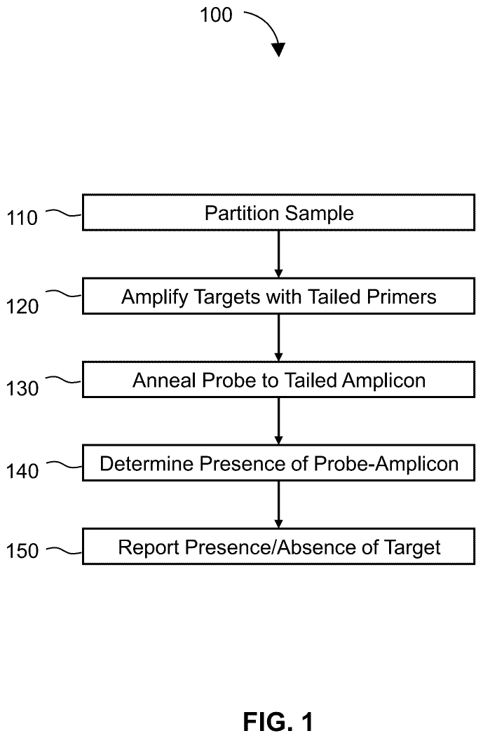

FIG. 1 diagrams steps of a method 100. The method 100 includes partitioning 110, into a plurality of aqueous partitions, a sample that includes at least two different targets (e.g., nucleic acid variants). The targets are amplified 120 using an amplification mix containing tailed primers and universal probes. During or after amplification, one or more of the universal probes anneals 130 to the tail of a tailed amplicon. Each universal probe is labelled with a respective color. A number of the plurality of partitions in which a universal probe is bonded to an amplicon is determined 140 by detecting light of the respective color from the partitions. Finally, the presence or absence of each target is reported 150 based on the number of partitions in which one or more probe is bound to an amplicon (indicating the presence of a target).

One of ordinary skill in the art will recognize that the number of targets to be detected depends on the specificity of target-specific primers and the resolution of clusters in radial multiplexing. In some embodiments, the methods of the invention can be practiced for the detection of one, two, three, four, five, six, seven, or more targets. Specifically, in some embodiments, the number of targets is three or more, four or more, six or more, or more than six targets. Further, one of ordinary skill in the art will recognize that the presence or absence of more targets can be determined when more color channels are analyzed. Accordingly, in some embodiments, data from two, four, six, or some other number of color channels is collected and analyzed pursuant to the disclosed methods. In some embodiments, the methods may be used with a series of n color channels to detect for >n variants.

The amplification mix includes reagents for PCR, such as at least one primer pair, polymerase, dNTPs, any cofactors or ions, and a set of “universal” probes, such as molecular beacons or fluorescent hydrolysis probes. The primer pairs are specific to the targets to be detected and include a “universal” tail. Accordingly, amplification provides tailed amplicons with a tail. The PCR synthesized tail portion is reactive with one or more of the “universal” probes.

It is envisioned to be within the scope of this disclosure that a single target may be amplified using more than one primer pair. Accordingly, the resulting tailed amplicon may anneal to more than one “universal” probe. It is also envisioned that multiple targets may be amplified with the same primers, or primers having the same tail sequences, but in different relative quantities. In these situations, the blend of probes binding to the amplicon tails would vary allowing detection of each target separately, even if they were amplified using different primers that have the same tail sequences.

The set of “universal” probes useful for the disclosed methods may be provided at a fixed or known concentration. The probes may include one or more of carboxyfluorescein (FAM), hexachlorofluorescein (HEX), cyanine5 (CY5), cyanine5.5 (CY5.5), carboxy-rhodamine (ROX), a fluorescent oligonucleotide dye with 594 nm adsorption (ATTO590), others, and any combination thereof. In some embodiments, the set of “universal” probes includes one probe for each of the six above. In some embodiments of the method, the set of “universal” probes may be used to detect targets in more than one assay without adjusting the probes. Accordingly, different from target-specific probes, the probes need not be modified or engineered for each target. Rather, the primers are engineered for each target and their concentration adjusted, if necessary, to effectively produce separate radial clusters. Some embodiments use super-selective primers, e.g., that include an anchor, a bridge, and a foot. Super-selective primers are exemplified in WO2014/124290, which is incorporated herein by reference in its entirety. Super-selective primers generally include single-stranded DNA oligonucleotides that possess three functional segments: the 5′ end is an anchor sequence that is virtually identical to the sequence of a conventional PCR primer, in that its length and sequence are chosen to selectively bind only to DNA templates of interest under the relatively high annealing temperature of the PCR assay; the 3′ end is a relatively short foot sequence that forms a perfectly complementary hybrid with an intended mutant target sequence, but that forms a thermodynamically less stable hybrid with a mismatched closely related sequence; and a central bridge sequence that is chosen by the primer designer to not be complementary to the intervening sequence in the DNA template that is located between the anchor target sequence and the foot target sequence. Moreover, the sequence of the bridge is chosen to not be complementary to any other sequence that may occur in the sample. Therefore, under PCR annealing conditions, the bridge sequence in the super-selective primer and the intervening sequence, which is located in the template strand to which the super-selective primer binds, form a single-stranded bubble that effectively separates the target-specific binding function of the relatively long anchor sequence from the mutation-selective function of the relatively short foot sequence. In embodiments of the invention, any primer pair may have either or both members be a super-selective primer and either or both members may have a 5′ universal tail. In some embodiments, the bridge sequence can function as the universal tail sequence and represent the universal sequence that the color probe will eventually hybridize upon amplicon PCR.

In some embodiments of the method, the set of “universal” probes may be used to detect targets and that same set of probes may be used in multiple different assays, e.g., without adjusting the relative concentration of the probes. For example, these reagents (e.g., amplification mix) may be loaded into a droplet-digital PCR (ddPCR) system, such as the system sold under the trademark QX600 by Bio-Rad Laboratories, Inc. The system may flow the aqueous sample at a preferred dilution through a microchannel to a junction where an aqueous fluid containing the amplification mix is added, downstream of which the aqueous mixture meets a cross flow of an immiscible carrier fluid such as a fluorinated oil. The aqueous reaction mixture breaks off into monodisperse water-in-oil droplets at the junction under co-flow conditions with the oil. Due to the preferred dilution, each droplet will contain zero or a small number of template molecules of nucleic acid from the sample. For example, zero, one, two, or three molecules per droplet may be common. The dilution can be calculated from a reading of nucleic acid quantity in the sample (e.g., optical density) and average fragment length (which may be a known result from sample processing or determined by, e.g., a gel). A surfactant (such a fluorosurfactant) may be added to promote droplet stability. Those aqueous droplets flow down a channel, surrounded by the oil, and may be collected in a suitable vessel such as a well of a 96-well plate. (In some such examples, the droplets are the partitions and collection into wells facilitates rapid heating of droplet contents or thermocycling.) On the droplet-digital PCR system, the plate may be subject to thermocycling such that the approximately tens of thousands of droplets in the well experience thermocycling conditions. Template molecules in the droplets are amplified by means of the primers, polymerase, and co-factors. During amplification, hydrolysis probes anneal to their targets (or amplicons thereof) when those targets are present and the probes are hydrolyzed by the polymerase, releasing fluorophores into the droplets. In other embodiments, during amplification, molecular beacon probes anneal to their targets (or amplicons thereof) when the targets are present resulting in a physical separation of the fluorophore from the quencher which are attached to the same probe molecule.

After amplification, the digital PCR system can load the droplets-spaced apart by oil-into a readout channel and flow the droplets past a detector. Preferred embodiments of the system read two optical channels (Channel1 and Channel2) during one readout operation. For example, channels 1 and 2 may read FAM and HEX, or other suitable dyes. Any fluorescence in either channel from each partition is plotted, forming a dot on a color plot. Targets can be distinguished in the multiplex assay by their clusters on the color plot.

In some embodiments, cluster detection is performed by software, and the variant makeup of the sample can be called by software that models Poisson distribution of variants into partitions during dilution and partitioning to identify the concentration of variants in a sample most likely to give the observed clusters.

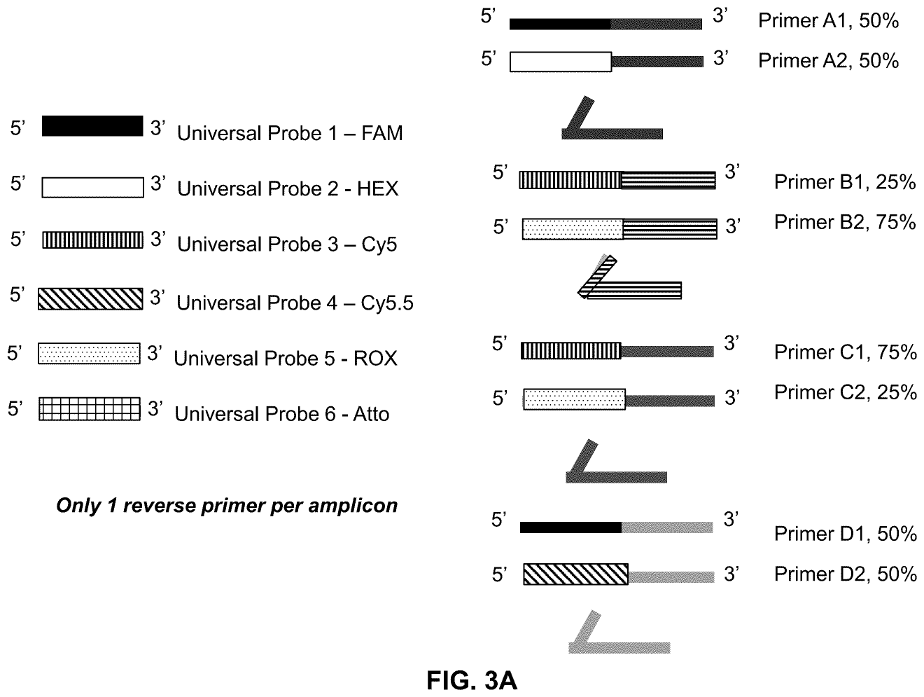

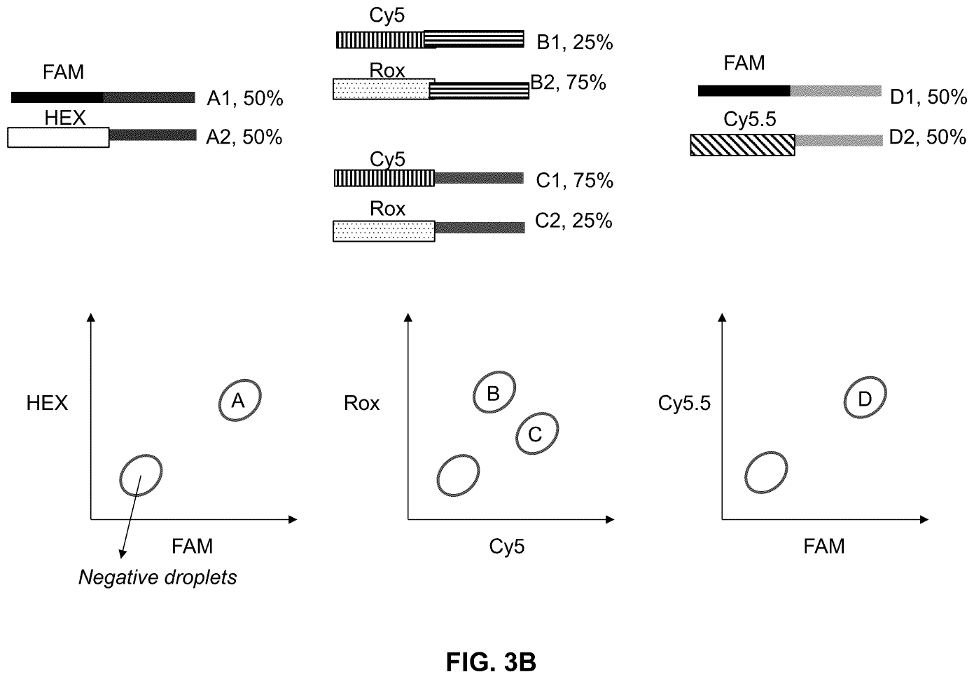

FIG. 3B illustrates clusters that have been identified on 2D plots in 6 channels, as well as the probes (drawn above each plot) used in generating that plot. In the simplified diagram, the points generated by fluorescent readout from each partition is not drawn but the clusters of points are represented by oval outlines, each labeled with the variant that gives rise to that cluster. Note that the cluster labeled “negative” represents partitions for which intensity readings are zero as relevant to the assay. The plots illustrate approaches to digital multiplexing, here referring generally to the detection of multiple different targets in a single digital assay readout or operation.

Digital generally refers to detection methods whereby any given target is detected as present or absent in a partition. Positive results, indicating the presence of the target, are agnostic as to the quantity of the target (single/multiple), only that the target is present. The polymerase chain reaction (PCR) has been used in digital assays that fall under the description of digital PCR, or dPCR. Digital PCR (dPCR) provides precise, highly sensitive quantification of nucleic acids. Digital PCR builds on conventional PCR amplification and fluorescent-probe-based detection methods to provide highly sensitive absolute quantification of nucleic acids without the need for standard curves. In the disclosed digital PCR Systems (dPCR) systems and methods, a PCR sample is partitioned into a large number (tens of thousands) aqueous partitions such as droplets. The partitions are provided with amplification reagents plus universal probes and thermocycled.

For a partition that contains a target molecule, thermocycling produces tailed amplicons. The universal probes anneal to the tails of the amplicons and generate fluorescence. Preferred embodiments use molecular beacons that match the tails of the primers and have nothing to anneal to until the primers are copied into amplicons with the reverse complement of the primer tail at the 3′ end of the amplicon, where the molecular beacon then binds to the 3′ tail of the amplicon. Other embodiments use fluorescent hydrolysis probes (e.g., TAQMAN branded probes) that match the primer tails, anneal to 3′ amplicon tails produced through PCR, and get digested by the 3′-5′ exonuclease activity when the second copy of the template is being synthesized during PCR. Whatever probe is used, droplets that contain a target of interest generate fluorescence as a result of amplification. A dPCR instrument or camera and microscope connect to a computer with analysis software to detect fluorescence from droplets. Each color of fluorophore can be said to be detected in its own dedicated channel of the detection system. Each partition may be read in each relevant channel, wherein partitions containing target sequence are detected by fluorescence and may be scored as positive, while partitions without fluorescence may be scored as negative. Poisson statistical analysis of the numbers of positive and negative droplets yields absolute quantitation of the target sequence.

In one embodiment, three targets may be detected using six probes. In this example, a first target is amplified with a set of primers which provide first amplicons; the forward primer comprises a tail that encodes, by its reverse complement, a binding site for a first probe to produce a first color and the reverse primer comprises a tail that encodes, by its reverse complement, a binding site for a second probe to produce a second color. Similarly, a second target is amplified with a second set of primers to provide second amplicons; the second forward primer comprising a tail that encodes for a third probe binding site to produce a third color and the second reverse primer comprising a tail that encodes for a fourth probe and fourth color. A third target is amplified with a third set of primers to provide a third amplicon; the third forward primer comprising a tail that encodes for a fifth probe and fifth color and the third reverse primer comprising a tail that encodes for a sixth probe and sixth color. In this way, the first amplicon is detected by the presence of both the first color and the second color. Likewise, the second amplicon is detected by the presence of both the third and fourth colors, and the third amplicon is detected by the fifth and sixth colors. Partitions that contained only a single color could be identified as likely the result of off-target amplification products and not the true amplicon of interest. Likewise, partitions that contained mixtures of colors not consistent with the target amplicons could also be distinguished from the true amplicons and can be further characterized as either containing mixtures of targets (e.g., target 1 and target 2) or off target products based on which colors are detected and/or the probability of multiple target co-occupancy. The problem of primer dimers and other off target effects common to many multiplex reactions are greatly reduced as single color or incorrect pairings, such as color 1 and color 4 or color 1 and color 5 can be distinguished from the correct pairings of color 1 with color 2, color 3 with 4 and color 5 with 6. Additionally, even when partitions are potentially occupied by more than 1 of the targets, the respective frequencies of the correctly paired colors can be used to estimate the frequency of target amplicons and non-target products which generate signals. The inventors recognize that the exemplary detection method described above can be extended to detect many more targets if desired.

One typical limitation with the universal probe approach is that signal levels are typically low when using non-cleaved probes unless the tailed primer is provided at a limiting concentration relative to the corresponding untailed “reverse” primer. See EP4038197 and EP1468114, incorporated by reference herein. This could limit the above 3 targets using 6 probes example to only cleavable probe designs, but non-cleavable probes have several advantages including lower cost and greater stability.

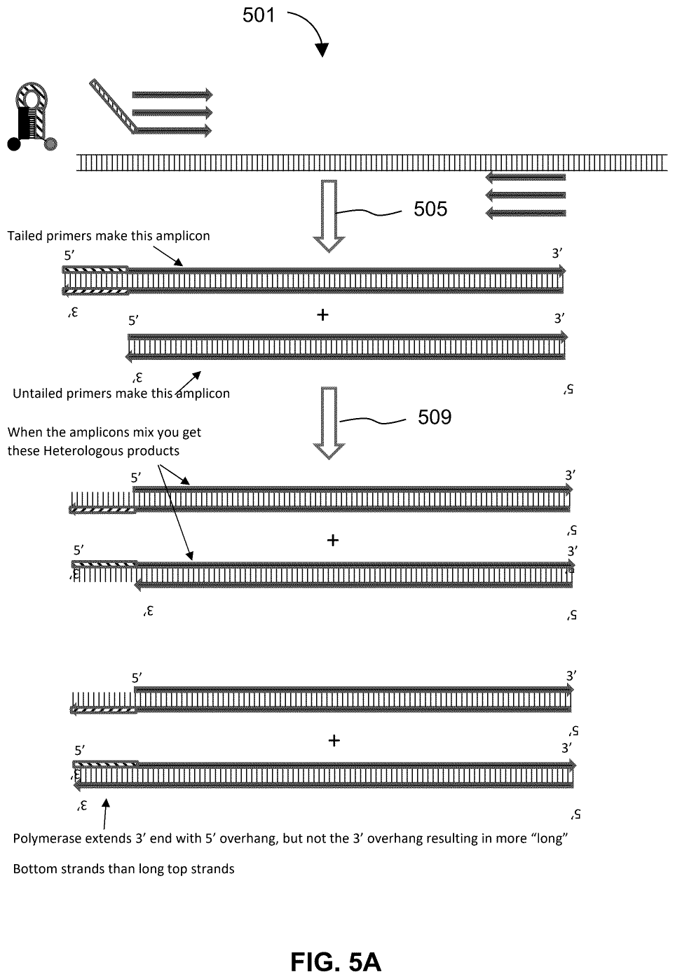

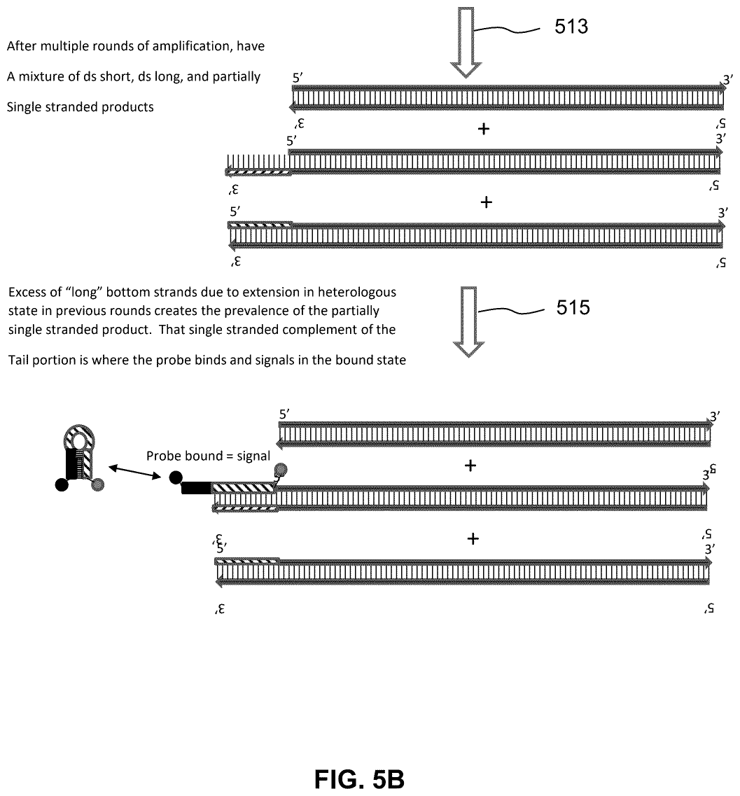

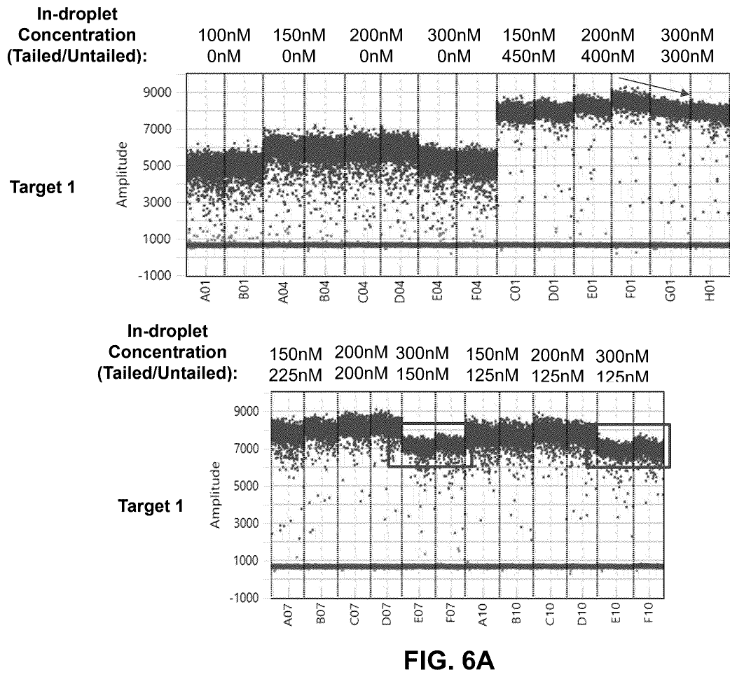

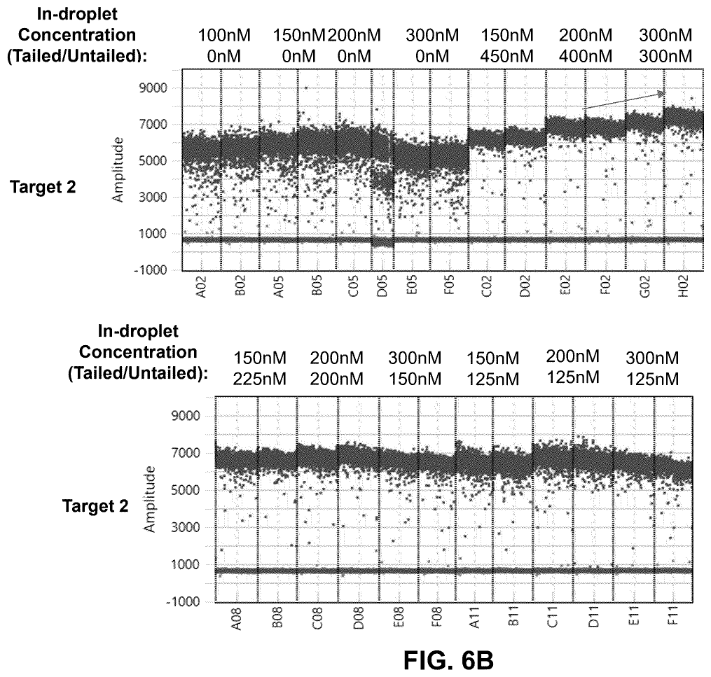

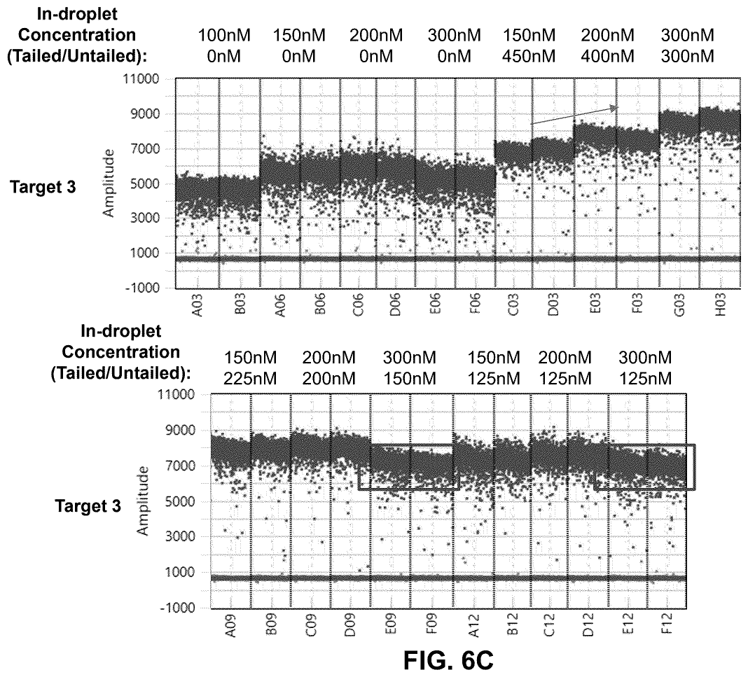

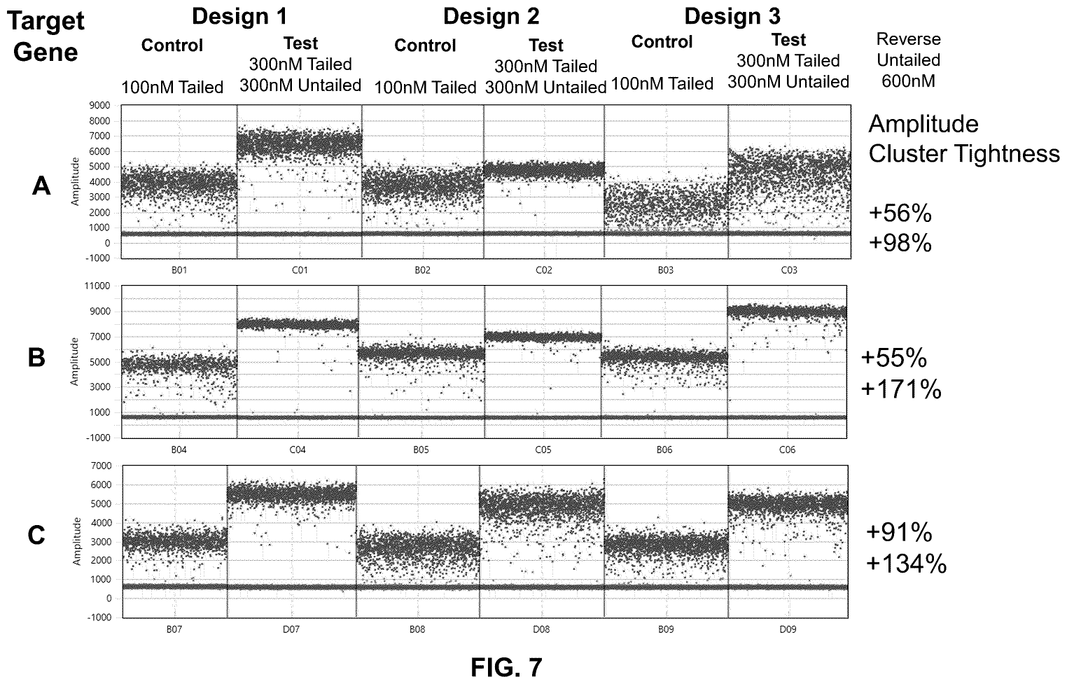

Also disclosed herein is a method to remove those limitations. Using mixtures of tailed and untailed primers to generate improved detection using universal probes without the need for cleavage of the probe or primer limiting the “forward” primer. For example, in the simple system where a first tailed forward primer is paired with an untailed reverse primer to generate first amplicons targeting a first target where the tail of the forward primer encodes a first probe binding site, it is often advantageous to substitute a mixture of tailed and untailed first forward primer rather than just the first tailed forward primer when amplifying by PCR. The tailed and untailed first forward primer are identical or substantively similar in the portion of the sequence that binds to the target sequence. In this way, both the first tailed and first untailed primer amplify the target of interest together with the reverse primer. The resulting amplicons share sequence identity other than the longer tail and complement section on the tailed amplicon. After denaturation (e.g., during PCR), complementary strands from the two amplicons can cross hybridize at the annealing temperature resulting in heterologous products that differ in length on one strand. Under PCR conditions, the heterologous products with a 5′ overhang is be extended to fill in the overhang, but those with a 3′ overhang will not. The result of this through several rounds of PCR will be partial or complete conversion of the “bottom” strands to the longer length which ultimately results in an excess of “long” bottom strands relative to long top strands, and thus the longer bottom strands being paired with shorter top strands. This disproportionate pairing results in a partially single stranded molecule wherein the single stranded portion corresponds to the complement of the tail of the tailed primer, and this is also the portion of the molecule that binds to the universal probe (see FIGS. 5A-5B). This mixing of tailed and untailed primers can result in improved assay performance including higher signals and tighter positive clusters and also removes the need for primer limiting the PCR reaction (see FIGS. 6A-6C and 7).

Accordingly, in some embodiments, a mixture of tailed and untailed forward primers is used. In some embodiments, a mixture of tailed and untailed reverse primers is used. In some embodiments, a mixture of tailed and untailed forward primers and tailed and untailed reverse primers is used. In some embodiments, the mixture has a ratio of tailed:untailed primers in a range of about 3:1 to about 1:3, about 2:1 to about 1:3, about 1:1 to about 1:3, about 2:1 to about 1:2, about 1:1 to about 1:2, or about 1:1. When present, the ratio for forward primers and/or reverse primers may be independently selected from these ranges.

A preferred embodiment utilizes mixtures of tailed and untailed primers for both the forward and reverse primer sets. In this way, the preferred non-cleaved probes can generate good signals for multiple configurations of the tailed primers; both forward and reverse tails can encode for the same probe, or the forward tail and reverse tail can encode for different probes (e.g., a first color for the forward primer tail and a second color for the reverse primer tail), or as described herein either the forward or reverse or both the forward and reverse primer tails may consist of mixtures of more than one tail sequence encoding for more than one probe in order to generate a signal comprised of multiple known color combinations.

An additional embodiment utilizes a mixture of tailed and untailed primers in quantitative PCR (qPCR). As described above, the mix of tailed and untailed primers results in an assay that is not primer limited. Accordingly, the assay will generate fluorescent signals in a fashion which is more readily used to quantify the amount of target in a sample (as in qPCR).

Concepts relevant to digital PCR were described in 1992 by Sykes et al., who recognized that the combination of limiting dilution, end-point PCR, and Poisson statistics could yield an absolute measure of nucleic acid concentration (albeit only by multiple replicates of a multi-step serial dilution). See Sykes, 1992, Quantitation of targets for PCR by use of limiting dilution, BioTechniques 13:444-449, incorporated by reference. A method was developed whereby a sample is diluted and partitioned to the extent that single template molecules can be amplified individually, each in a separate partition, and the products detected using fluorescent probes. See Vogelstein, 1999, Digital PCR, PNAS 96:9236-9241, incorporated by reference. Those teachings may be employed here in the dPCR assays of the disclosure.

Digital PCR improves upon the sensitivity of qPCR and provides for the detection of rare events such as single-nucleotide mutations in a population of wild-type sequences. A dPCR assay involves diluting a sample to promote isolation of single template molecules into partitions (e.g., diluting to a degree that each partition, on average, contains either zero or one template even while some small numbers of partitions may have duplets or multiples of templates, which are negligible to final results). By minimizing the effects of competition between targets, digital PCR overcomes the difficulties inherent to amplifying rare sequences and allows for sensitive and precise absolute quantification of nucleic acids. Preferred embodiments of digital PCR involve sample partitioning—the division of a sample into discrete subunits prior to amplification by PCR. The sample is prepared in a manner similar to that for real-time PCR but is then separated into e.g., thousands of partitions, each ideally containing either zero or one (or, at most, a few) template molecules. Each partition behaves as an individual PCR reaction and, as with real-time PCR, fluorescent probes are used to identify amplified target DNA. Each partition can then be readily analyzed after amplification to determine whether or not it contains the target sequence. Samples containing amplified product are considered positive (1, fluorescent), and those without product, and thus with little or no fluorescence, are negative (0). The ratio of positives to negatives in each sample is the basis of quantification.

Methods preferably include diluting the sample so that each partition receives a limited number of target molecules, such as zero, one, two, sometimes three, and a very small number of four or more. The dilution can be calculated so that a majority of partitions receive a target number (e.g., zero or one) of targets. Each target molecule will serve as a template for generation of amplicons in the presence of fluorescence probes.

Partitioning can be accomplished by any suitable mechanism and any suitable type of aqueous partition may be used for digital PCR. Exemplary suitable partitions include droplets, wells in a plate, or other fluid portioning structures. For example, the partitions may be wells, cavities, pockets, or openings in a pico-, nano-, or microtiter plate or substrate, or fluidic harbors (see, e.g., US Pub 2010/0041046 A1, incorporated by reference). The partitions may be wells in a multi-well plate such as a 96-well plate, 384 well plate, a 1536 well plate, a 3456 well plate, or a 9600 well plate. The partitions may be separate chambers (see, e.g., 2021/0178395 A1, incorporated by reference). The partitions may be distinct regions defined within a fluidic device (see, e.g., 2020/0269248 A1, incorporated by reference). The partitions may be aqueous slug in, and occluding, channels of fluidic device, optionally separated by an immiscible fluid. In certain embodiments, the partitions are a plurality of droplets that are formed essentially simultaneously by mixing together and vortexing an aqueous fluid and oil. In preferred embodiments, the partitions are droplets of an emulsion such as a water-in-oil (W/O) emulsion or a water-in-oil-in-water (W/O/W) emulsion.

Preferred embodiments use aqueous partitions in an immiscible liquid, e.g., slugs, capsules, or droplets surrounded or separated by immiscible carrier fluid such as an oil within a microfluidic device. Aqueous droplets in an immiscible carrier liquid may be formed by microfluidic handling. A microfluidic device may use channels to mix samples and reagents and form droplets in an immiscible carrier fluid. Droplets may be formed within a partitioning section or subunit of a digital PCR instrument or system that uses, e.g., channels to flow an aqueous mixture into an intersecting stream of carrier oil.

The unique sample partitioning step of digital PCR, paired with Poisson statistical data analysis, allows higher precision than traditional PCR and qPCR methods. Accordingly, digital PCR is particularly well suited for applications that require the detection of small amounts of input nucleic acid or finer resolution of target amounts among samples, for example, rare sequence detection, copy number variation (CNV) analysis, and gene expression analysis of the rare targets.

Techniques used in digital PCR include PCR amplification on a microfluidic chip. See Ottesen, 2006, Microfluidic digital PCR enables multigene analysis of individual environmental bacteria, Science 314:1464-1467, incorporated by reference. Other systems involve separation onto microarrays (Morrison, 2006, Nanoliter high-throughput quantitative PCR, Nucleic Acids Res 34: e123, incorporated by reference) or spinning microfluidic discs (Sundberg, 2010, Spinning disk platform for microfluidic digital polymerase chain reaction, Anal Chem 82:1546-1550, incorporated by reference) and droplet techniques based on oil-water emulsions (Hindson, 2011, High-throughput droplet digital PCR system for absolute quantitation of DNA copy number, Anal Chem 83:8604-8610, incorporated by reference), as in droplet digital PCR (ddPCR) systems such as the ddPCR system sold under the trademark QX200 by Bio-Rad Laboratories, Inc. (Hercules, CA).

In such digital assays, each reaction mixture (e.g., partition) is interrogated for the presence or absence of target. The detection of target may be performed using optical density, intercalating dyes, ethidium bromide, change in pH, release of pyrophosphate, or any other suitable method for detecting a target. Preferred embodiments of the invention use fluorescent universal probes. Preferred fluorescent universal probes are molecular beacons. Molecular beacons, or molecular beacon probes, are oligonucleotide hybridization probes that can report the presence of specific nucleic acids in homogenous solutions. Molecular beacons are hairpin-shaped molecules with an internally quenched fluorophore whose fluorescence is restored when they bind to a target nucleic acid sequence.

In preferred embodiments, a molecular beacon probe is between 20 and 30 nucleotides long in which about middle target-binding segment is complementary to the target DNA or RNA and do not base pair with one another, while the terminal nucleotides are complementary to each other and form a hairpin. Typical molecular beacons may have 4 parts: (1) target binding segment or loop, an 18-30 base pair region of the molecular beacon that is complementary to the target sequence; (2) stem formed by the attachment to both termini of the loop of two short (e.g., 5 to 7 nucleotide residues) oligonucleotides that are complementary to each other; (3) fluorescent dye covalently attached at one location, e.g., 5′ end; and (4) quencher covalent attached at a second location, e.g., 3′ end. When the beacon is in closed loop shape, the quencher resides in proximity to the fluorophore, which results in quenching the fluorescent emission of the latter. Remembering that universal-tailed primers of the disclosure preferably match the universal probes in sequence and in sense, only after those primers are copied during PCR do the amplicons include segments that are reverse complement of the target binding segment of the probe. When those amplicons are produced, probes anneal, or hybridize, to the amplicons. The duplex formed between the nucleic acid and the loop is more stable than that of the stem because the former duplex involves more base pairs. This causes the separation of the stem and hence of the fluorophore and the quencher. Once the fluorophore is no longer next to the quencher, illumination of the hybrid with excitation light results in the fluorescent emission. The presence of the emission reports that the event of hybridization has occurred and hence the target nucleic acid sequence is present in the test sample. PCR polymerases may be exonuclease minus when using molecular beacons and probes can remain intact while generating fluorescence.

Other universal probes may be used in methods, systems, and kits of the invention. For example, some embodiments use universal fluorescent hydrolysis probes similar to the probes sold under the trademark TAQMAN by Thermo Fisher Scientific (Waltham, MA). As used in the invention, hydrolysis probes are preferably antisense to, and bind to, 5′ tails of the universal primers. After a tailed primer is extended, Taq polymerase extends a reverse primer to copy that extension product. When the polymerase reaches an annealed probe (in the 3′ tail), the 3′-5′ exonuclease activity of Taq pol digests the hydrolysis probe, releasing the fluorophore from the quencher. Hydrolysis probes include an oligonucleotide backbone that anneals in a sequence specific manner to a target of that probe, plus a fluorophore and a quencher. Methods may include thermocycling the droplets within a reaction tube or well of a plate. During amplification with polymerase, exonuclease activity of the fluorophore digests the oligonucleotide backbone of any bound hydrolysis probe, separating the fluorophore from the quencher, allowing the fluorophore to fluoresce during a readout step. To read fluorescence, methods may include flowing the droplets (e.g., one-at-a-time) past a detector (and optionally a light source for excitation of fluorophores).

Preferred ddPCR systems read two channels (or two colors of fluorescence) together at a time. For example, some ddPCR system flow droplets past two color detectors and, for each droplet, two colors are read simultaneously or in close temporal sequence. Some such platforms have multiple, e.g., six, color channels, but typically read two colors at a time from each partition to generate results such as the color plots of FIG. 3B. For example, the center plot of FIG. 3B illustrates radial multiplexing, which can be seen because a number of different targets (B and C) are each independently detected from the two color readings.

When the multiple variants are amplified in partitions, the tailed amplicons will anneal to one of the probes, which will fluoresce. Each partition that includes a target will absorb fluorescence excitation light, which may be provided by a light source such as an LED within a ddPCR system. Such a system typically includes at least two channels, sometimes referred to as a color channel, that includes a detector such as a photodiode to detect and record optical signal from each partitions. As each partition is read (e.g., as a droplet flows past the photodiodes of two channels) the amplitude of fluorescence from that partition is recorded. The recorded amplitude of the two colors of fluorescence (e.g., HEX and Cy5) may then be plotted. Each partition will provide one of the points on a color plot. In fact, from the two (or more) detection channels of a dPCR system, there are software packages available that create 2D plots showing the clusters and even detect and discriminate the clusters and implement a model based on Poisson statistics to provide quantities of the variants in the sample based on the observed data. One such software packages is the dPCR analysis software sold under the trademark QUANTASOFT by Bio-Rad Laboratories, Inc.

Other approaches to “calling” a sample (reporting the presence or absence of variants for which probes were introduced) include the use of available software packages such as the software package named “dPCR Cluster Predictor” (dPCP), an R package and a Shiny app for automated analysis of up to 4-plex dPCR data. Such a software package can analyze and visualize data generated by multiple dPCR systems carrying out accurate and fast clustering not influenced by the amount and integrity of input of nucleic acids. See De Falco, 2023, Digital PCR cluster predictor: a universal R-package and shiny app for the automated analysis of multiplex digital PCR data, Bioinformatics 39 (5): btad282, incorporated by reference.

As discussed, methods of the invention may be used with a universal probe blend for digital multiplex detection of multiple targets with two-channel readout at a time. For example, five targets may be read in two color channels. Some systems and platforms may use more than two color channels. In some embodiments, four or six or more color channels are used. In some embodiments, the color channels are used two at a time during readout. In some embodiments, all color channels are used simultaneously during readout. For example, the method may include reading the sample for at least seven variants using at least six colors, wherein the detecting step reads two of the six colors, in two channels, at a time.

Such a multiplexing assay may be used for any suitable purpose in research, medicine, inquiry, diagnostics, or any other field of endeavor. For example, the depicted methods may be used for the analysis of tumor nucleic acids. In some examples, a patient may have or have had a tumor. In some such cases, an analysis may have been previously performed on tumor DNA from the patient (e.g., from a tumor biopsy or tumor slice mounted on a slide and, e.g., formalin-fixed and paraffin embedded). The tumor DNA may have been sequenced by, e.g., next-generation sequencing (NGS) to generate tumor sequence reads that have been analyzed by comparison to matched normal reads or a reference to identify tumor-specific mutations. A set of (patient-specific) PCR primer pairs that flank those mutations may be designed (e.g., using primer design software known in the art) and synthesized such that each primer pair has at least one primer with a 5′ universal tail. The universal tails are preferably complementary to universal fluorescent probes that are separately provided and specific to each primer pair. In such cases, methods of the invention may be used, at some later time (such as after treatment), to analyze a sample from that patient for tumor DNA. For example, a liquid biopsy sample such as blood draw may be analyzed for cell-free DNA or to capture, and extract nucleic acid from, circulating tumor cells (CTCs), in either case to obtain a sample that potentially includes tumor DNA. That sample may be partitioned along with the universal probes and the patient-specific primer pairs to interrogate, by dPCR, the sample for the presence of each of the tumor-specific mutations. Each mutation is interrogated with a blend of probes specific for that mutation. In but one illustrative example, if there are five mutations, there can be five primer pairs, and to read each mutation in a single instrument run using two color channels, each primer pair can be used with a characteristic quantity of two 5′ universal tails with binding sites for two probes labelled with two dyes specific to the to color channels, as shown in Table 1.

| Target | % First Tail | % Second Tail |
|---|---|---|
| Variant 1 | 100 | 0 |
| Variant 2 | 75 | 25 |
| Variant 3 | 50 | 50 |
| Variant 4 | 25 | 75 |
| Variant 5 | 0 | 100 |

The values in Table 1 may be varied by any amount provided that, preferably, each row is unique. With a 2-channel instrument, any number of variants can be read including 0, 1, 2, etc., further including 5 as shown in the example, and 6, 7, 8, 9, etc. The sample is diluted into some number (e.g., thousands or tens of thousands or more) of partitions, where the dilution ensure that most partitions include either zero or one molecule of nucleic acid (which can be done by DNA extraction, e.g., using a commercially-available kit or column, into a tube or cuvette, using e.g., spectrophotometry to estimate quantity of nucleic acid, and diluting to a concentration at which the partitions with their intended number and volumes will receive the mostly 0 or 1 molecule each). In some embodiments the sample is diluted where the partitions ensure that some partitions include either zero or a limited number of molecules of nucleic acid. In said embodiments, Poisson statistics can be used to calculate the concentration of nucleic acids. The partitions are also provided with the primer pairs, universal probes, and PCR reagents. The partitions are thermocycled and read in two optical channels (optionally more than 2). Each partition will emit a signal in the two channels (negative signals are not typically zero due to auto-florescence and other stochastic factors). Each partition may be plotted on a 2D plot, with an axis for each color. Each tumor-specific mutation, if present, will give rise to a distinct cluster of points on the 2D plot. Those clusters are distinct at least in the sense that one could draw radii emanating from an origin such that each cluster essentially appears to be centered on its own radius. Those clusters are preferably detected by software and it need not be the case that a visual plot has perfect radial separation. Rather, obtained results tend to show what appears visually to be good radial separation and analytical software reliably detects each target, including when assays have been run with known spiked-in samples. As an aside, it is noted that the origin need not be the (0,0) point on the axes for color, but can be assigned e.g., to the geometric center of the double-negative cluster (which is not typically at optical (0,0) due to autofluorescence or other experimental noise). In the manner described, each target is separately detected in a single 2-channel operation, i.e., multiplexed, and that approach to multiplexing is here reasonably described as radial multiplexing.

Each partition may be read for fluorescent signal from the universal probes, using multiplexing to read any arbitrary number of the tumor-specific mutations in 2 color channels at a time (e.g., in a 2-channel or 6-channel dPCR instrument). The results may be presented in any suitable fashion such as by plotting points representing fluorescence from each partition on a 2D plot for each pair of channels that is read simultaneously.

Thus, it can be seen that method of the invention may use radial multiplexing, which can be implemented by reading two colors from each of a plurality of partitions, plotting intensity of the two colors on a 2D plot with axes for intensity of each color, and identifying clusters of points on the plot. Each cluster will typically be found essentially lying along distinct radius extending from a cluster corresponding to double-negative (no significant fluorescence of either color) partitions. In radial multiplexing, clusters corresponding to distinct targets in the sample are distinguishable according to their different radial directions from the double negative cluster. Clusters can further be distinguished based on radial distance. Thus, five or more targets can be distinguished by providing mixtures of probes with varying amounts of the two fluorescent reporters interrogated in the two channels. Other methods of multiplexing such as fluorescence intensity multiplexing are within the scope of the disclosure. For radial multiplexing, the reporting step may include plotting the amounts of the first color and the second color detected from the partitions as points on a graph and identifying clusters of the points on the graph corresponding to the presence of any of the three variants. The quantities of the respective targets in the sample may be determined by a Poisson model of templates into the partitions that would generate the observed pattern of clusters.

Embodiments of the invention are described and illustrated using radial multiplexing. However, other multiplexing techniques are within the scope of the invention. For example, all probes may have a unique color and each variant may be read in its own respective color channel. Other embodiments involve the use of fluorescence intensity multiplexing, which may also be referred to as amplitude multiplexing. Other embodiments involve the use of a combination of multiplexing techniques, including radial, single color, and intensity multiplexing.

Another approach to multiplexing may be referred to as computational multiplexing. Computational multiplexing may be performed without creating any 2D graph or plot or other such printable display. For computational multiplexing, fluorescent signal is read from each partition and stored or delivered to an analysis package (e.g., software) in for example a tabulated or data normal format. The analysis package may analyze the tabulated data by processes such as an analysis of variance or a principle component analysis. The software package may perform a K-means clustering analysis. Some approaches involve a combination of density-based spatial clustering of applications with noise (DBSCAN) (Ester, 1996, A density-based algorithm for discovering clusters in large spatial databases with noise, Knowl Disc Data Min 226-31, incorporated by reference) and the c-means algorithms optionally using a reference sample for classification of clusters with few elements. After discovery of the clusters e.g., DBSCAN, the analysis package may perform a calculation of centroid positions of the clustering, then perform a Cmeans analysis. Further detail and additional steps that may be performed are described as the software package “digital PCR cluster predictor” in DeFalco, 2023, Digital PCR cluster predictor: a universal R-package and shiny app for the automatic analysis of multiplex digital PCR data, Bioinformatics 39(5):btad282, incorporated by reference. Such an analysis package may also be provided by the software package sold under the trademark QUANTASOFT Analysis Pro Software by Bio-Rad Laboratories, Inc. (Hercules, CA). Clusters in 2-channel readings may also be identified by “data gridding”, which involves overlaying a grid on the raw results, transforming the gridded data into binary components, using a sliding window to take the average of the number of partitions in each grid coordinate, using k-means clustering to create initialization coordinates, and estimating boundaries for each cluster. Such data gridding approaches may use the software pipeline called “Calico” (Iterative clustering assisted by coarse-graining). The Calico software package is available as open-source from github and is described in Lau, 2021, Robust multiplexed clustering and de-noising of digital PCR assays by data gridding, Anal Chem 89(22):11913-11917, incorporated by reference. Other approaches to computational multiplexing including writing a new software program that performs cluster detection (“home-grown”).

Regardless of the analysis package (e.g., digital PCR cluster predictor, the QUANTASOFT analysis software, Calico, home-grown, others, or any combination thereof), an analysis package may be used to assign each partition to a location a 2D space (without necessarily displaying anything in a human-readable form). The analysis package can detect the clusters and “call” each as to which target it represents in the sample. Thus any arbitrary number of targets may be detected in multiplex, using universal probes, using 2-color channels (at a time) for read, with multiplexed detection being performed computationally, using an analysis package executed by computer hardware comprising at least one processor coupled to non-transitory memory.

Approaches to multiplexing according to this disclosure use universal probes for multiplex detection. Each instrument run will typically use 2, or 4, or 6 (or some other similar number) of such probes. The probes are “universal” in that they do not anneal to genomic sequences in the targets but instead bind to designed universal probe binding sites in primer tails or the reverse complement thereof produced during amplicon PCR. Each probe is present in excess, e.g., in nM concentration or in millions or billions clonal copies per partition. Each probe sequence is linked to one fluorophore, but each probe can be present as a mixture using two different fluorophores. The probes can be designed, ordered, synthesized, and/or stored “ahead of time”, i.e., independently of any detection assay. Probes may be provided in a reagent bottle or pouch, for example, that can be connected to or used to supply a dPCR instrument. Each instrument run involves the use of primer pairs that are designed and synthesized for the specific targets being interrogated in that assay. The primers may be simple DNA oligonucleotides (although any modified oligos may be used). The probes are preferably molecular beacons-DNA oligos linked to a fluorophore and a quencher, with a central target-binding segment or loop flanked by a pair of hairpin forming arms. The target-binding segment is universal, i.e., anneals to copies of 5′ tails present on primers in the primer pairs. The target binding segment substantially matches (i.e., has the same sense as) the 5′ targeting tails. When present among the primers and their 5′ targeting tails, the probes form a hairpin (there being nothing for the target-binding segment to anneal to), quenching the fluorophore by their proximity. When the primers are successfully used in amplifying their targets, copies of the targeting tails anneal to the target-binding segment of the probe, opening the hairpin, separating the quencher from the fluorophore, resulting in fluorescence from the universal probe.

FIG. 5A shows the first half of a method 501 using mixtures of tailed and untailed primers. The method 501 includes annealing 505 tailed and untailed primers to target nucleic acid. The tailed and untailed primers are annealed 505 and extended and the products are amplified 509 to generate amplicons, which mix to yield heterologous products. Polymerase extends 3′ end with 5′ overhang, but not the 3′ overhang resulting in more “long” bottom strands than long top strands.

FIG. 5B shows the second half of the method 501 using the mixtures of primers. The method 501 continues through multiple rounds of amplification 513. After multiple rounds of amplification, the method 501 has produced a mixture of double stranded (ds) short, ds long, and partially single stranded products. The method 501 preferably includes annealing 515 probe to target. Excess of “long” bottom strands due to extension in heterologous state in previous rounds creates the prevalence of the partially single stranded product. That single stranded complement of the tail portion is where the probe binds and signals, once in the bound state.

### EXAMPLES

Embodiments of the invention are identified below. One of ordinary skill in the art will readily understand that modifications to the disclosed examples are within the scope of this disclosure.

### Example 1

A set of universal probes is prepared containing six probes at fixed concentrations. Each probe is engineered to anneal to a specific 3′ tail of a target amplified with a tailed forward primer.

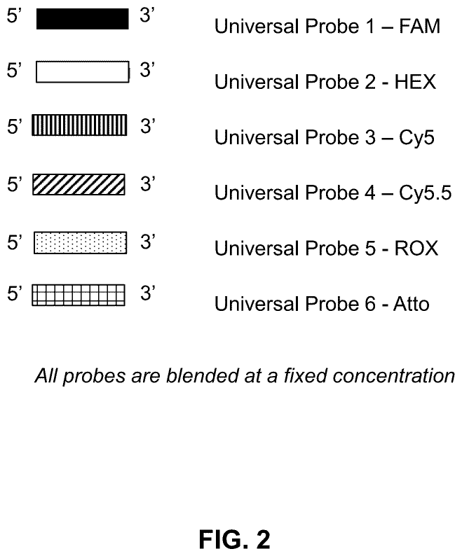

With reference to FIG. 2, for a six-color channel analysis, the probes may be as follows: FAM-tail sequence 1; HEX-tail sequence 2; Cy5-tail sequence 3; Cy5.5-tail sequence 4; ROX-tail sequence 5; Atto-tail sequence 6. In some embodiments, each of the set of probes is described as corresponding to 5′ tail sequence XX of primers. This may also be disclosed as Universal Probe XX. For the avoidance of doubt, universal probe XX is reactive with, and preferably only with, tail sequence XX.

As stated earlier, these universal probes may be blended at a single concentration. This blend may be commercialized to be used for multiple assays.

Referring to FIG. 3A, for this example, a sample containing four targets (A, B, C, and D) is analyzed to determine the presence or absence thereof. To achieve radial multiplexing, a blend of primers is constructed so that each target is amplified with more than one primer. The figure shows 12 primers (and 6 probes): two forward primers (A1 and A2) plus 1 reverse primer for target A (each reverse primer is drawn with a half arrowhead); target B is interrogated with forward primers B1 and B2 plus a reverse primer; target C has forward C1, forward C2, and a reverse; while for target D, the figure shows forward primer D1, forward primer D2, and a reverse primer. Each primer, as drawn, has a 5′ end tail color-coded according to its matching probe. Accordingly, the 6 depicted universal probes will each anneal to amplicons made when copying one of the 8 depicted forward primers.

In this example, target A is amplified to produce two amplicons. A first amplicon (A1) reacts with Probe 1 (FAM) and a second amplicon (A2) reacts with Probe 2 (HEX). The two amplicons (A1, A2) may be produced in relatively equal quantities.

Target B is amplified to also produce two amplicons. A third amplicon (B1) reacts with Probe 3 (Cy5) and a fourth amplicon (B2) reacts with Probe 5 (ROX). The primer concentrations are controlled such that B2 is produced three times more than B1 (25% B1, 75% B2). Target C is also amplified to produce two amplicons. A fifth amplicon (C1) reacts with Probe 3 (Cy5) and a sixth amplicon (C2) reacts with Probe 5 (ROX). The primer concentrations may be controlled such that C1 is produced three times more than C2 (75% C1, 25% C2). Target D is amplified to produce two amplicons. A first amplicon (D1) reacts with Probe 1 (FAM) and a second amplicon (D2) reacts with Probe 4 (Cy5.5). The two amplicons (D1, D2) may be produced in relatively equal quantities.

As identified above, the primers are blended at specific concentrations to achieve a desired location of a positive cluster in the color space once the universal probe mixture hybridizes to its reverse complement of the primer tail after PCR synthesis. When the combinations are the same, e.g., for Target B and C above, the different proportions deliver a variable color space location to provide differentiation. For example, in some embodiments, to achieve a 4plex of radial clusters, the depicted fluorescent probe mixture is used and four combinations of tailed primers are blended at specific concentrations to achieve differentiation.

Referring to FIG. 3B, three color plots are shown which report the four targets of the example. The left plot (FAM v. HEX) shows target A, the center plot (Cy5 v. ROX) shows both target B and target C, while the right plot (FAM v. Cy5.5) shows target D.

One of ordinary skill in the art will recognize that based on the ability to engineer target-specific primers for targets of interest, many more targets may be determined in other color plots, including targets with only a single reactive probe.

### Example 2

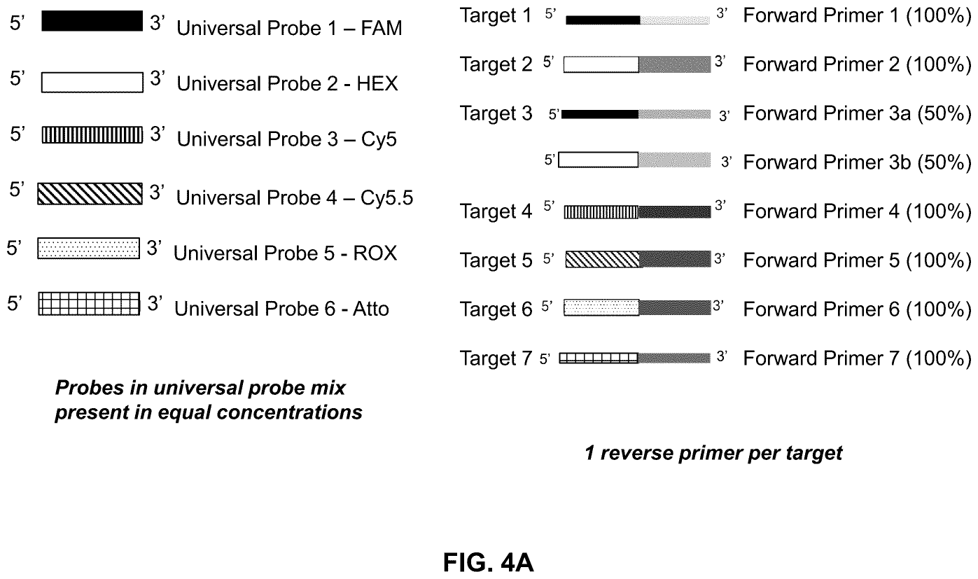

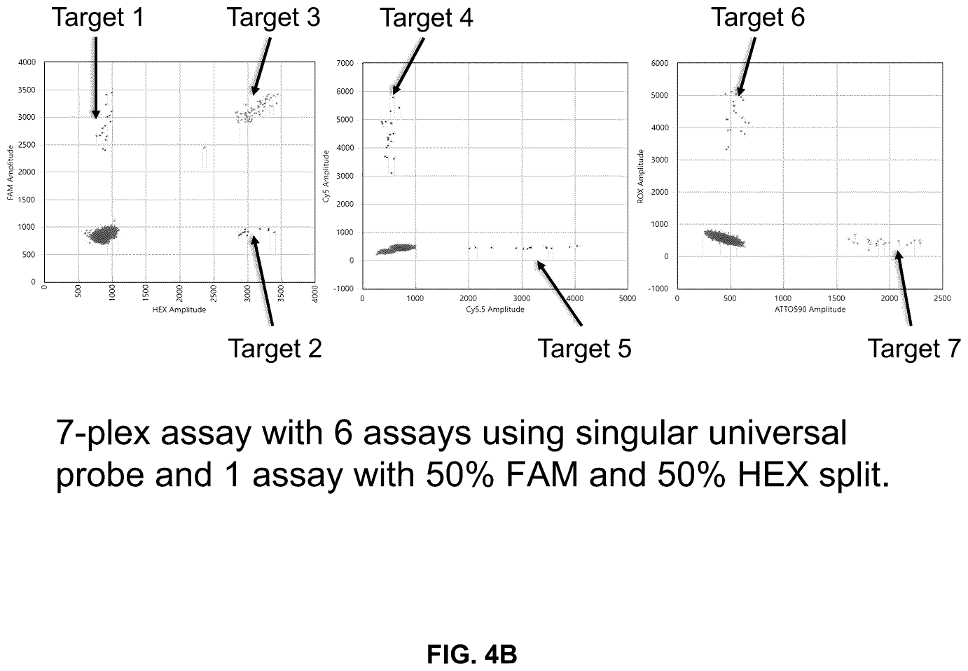

Referring to FIG. 4A, another example is illustrated regarding the detection of seven targets (1-7). The same blend of probes is used from Example 1.

To achieve radial multiplexing, a blend of primers is constructed so that each target is amplified with one or more primers. FIG. 4A indicates 15 primers (and 6 probes): one forward primer and one reverse primer for each of targets 1-2 and 4-7; and two forward primers and one reverse primer for target 3. Each primer, as drawn, has a 5′ end tail color-coded according to its matching probe. Accordingly, the 6 depicted universal probes will each anneal to amplicons made when copying one of the 8 depicted forward primers.

In this example, targets 1-2 and 4-7 are amplified to produce one amplicon. Each amplicon reacts with a single probe from the universal probe mix. The amplicon corresponding to target 1 binds to universal probe 1 labeled with FAM; the amplicon corresponding to target 2 binds to universal probe 2 labeled with HEX; the amplicon corresponding to target 4 binds to universal probe 3 labeled with Cy5; the amplicon corresponding to target 5 binds to universal probe 4 labeled with Cy5.5; the amplicon corresponding to target 6 binds to universal probe 5 labeled with ROX; and the amplicon corresponding to target 7 binds to universal probe 6 labeled with Atto.

In contrast, target 3 is amplified to also produce two amplicons. A first amplicon reacts with Probe 1 (FAM) and a second amplicon reacts with Probe 2 (HEX). The primer concentrations are controlled such that the amplicons are produced in approximately equal quantities (50% first, 50% second).

As identified above, the primers are used at specific concentrations to achieve a desired location of a positive cluster in the color space once the universal probe mixture hybridizes to its reverse complement of the primer tail after PCR synthesis.

Referring to FIG. 4B, three color plots are shown which report the seven targets of the example. The left plot (HEX v. FAM) shows targets 1-3, the center plot (Cy5.5 v. Cy5) shows targets 4-5, while the right plot (ATTO 590 v. ROX) shows targets 6-7.

### Example 3

A series of experiments were conducted to determine the effect of various concentration combinations of tailed and untailed forward primers. For these experiments, 600 nM of untailed reverse primer was used. The results across 3 targets are presented in FIGS. 6A-6C.

While different applications will require different outcomes, the combinations with the greatest amplitude and/or cluster tightness were preferred. As evident in FIGS. 6A-6C, increasing tailed primer concentration without untailed primer had a relatively small effect on amplitude and cluster tightness. In contrast, adding untailed forward primer resulted in significantly improved amplitude and/or cluster tightness across the three targets tested.

These results demonstrate the advantage in using a mixture of tailed and untailed forward primer rather than just the tailed forward primer when amplifying by PCR.

Referring to FIG. 7, the results of three test designs on three genetic targets is shown. In each of the 9 experiments, the test case (300 nM/300 nM tailed/untailed primer mixture) provided increased amplitude and cluster tightness over the control (100 nM tailed primer only). The average increases in amplitude and cluster tightness are shown in the right column.

Features described above as well as those claimed below may be combined in various combinations without departing from the scope of the invention. The following examples illustrate some possible, non-limiting combinations:

(A1) A target detection method for detecting one or more targets in a sample. The method may comprise partitioning a sample comprising, suspected of comprising, or being tested for a plurality of targets into a plurality of partitions. The targets are amplified with mixtures of primers in which a plurality are tailed primers to yield amplicons with tails. To detect the targets, universal probes that bind to at least one primer tail or a complement thereof in amplicons are used. Each of the universal probes is labeled with a respective label (e.g., a fluorescent color). A number of the plurality of partitions is determined in which one or more universal probes bound to a corresponding amplicon by detecting the corresponding label (e.g., light of the respective color) from the plurality of partitions. The presence or absence and/or quantity of each of the targets is reported in the sample based on the number of the plurality of partitions in which the universal probes bound to their corresponding amplicons.

(A2) For the method denoted as (A1), the sample: (a) comprises, is suspected of comprising, or is being tested for any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more, targets; or (b) comprises, is suspected of comprising, or is being tested for more than six targets; and the method includes detecting the presence or absence of each of the targets in two color channels (e.g., using two universal probes), four color channels (e.g., using four universal probes), five color channels (e.g., using five universal probes), six color channels (e.g., using six universal probes), seven color channels (e.g., using seven universal probes), eight color channels (e.g., using eight universal probes), nine color channels (e.g., using nine universal probes); and wherein each universal probe is linked to a respective label such as a fluorophore.

(A3) For the method denoted as (A2), the fluorophores include one or more of carboxyfluorescein (FAM), hexachlorofluorescein (HEX), cyanine5 (CY5), cyanine5.5 (CY5.5), carboxy-rhodamine (ROX), and a fluorescent oligonucleotide dye with 594 nm adsorption (ATTO590).

(A4) For the method denoted as any one of (A1) to (A3), each target is amplified using primers with universal tails, and optionally each primer is provided at a relative concentration specific to each target.

(A5) For the method denoted as any one of (A1) to (A4), a set of universal probes at fixed concentrations for each probe is used, for example a set of universal probes including any of 2, 3, 4, 5, 6, 7, 8, 9, 10, or more, universal probes is used, wherein optionally each universal probe includes a label such as a fluorophore, further optionally wherein the universal probes are used in combination with a set of tailed primers at a relative concentration specific to the target to be detected.

(A6) For the method denoted as any one of (A1) to (A5), for each target, the target is amplified using (i) a mix of forward primers each having a universal tail corresponding to a respective universal probe and provided at a relative concentration specific to that target, and (ii) a reverse primer.

(A7) For the method denoted as any one of (A1) to (A6), each target is detected as present or absent by: mapping the color detected from each partition into at least a 2-axis color space; and detecting a cluster for each target in the color space.

(A8) For the method denoted as any one of (A1) to (A7), the probes comprise molecular beacons or hydrolysis probes.

(A9) For the method denoted as any one of (A1) to (A8), the method further comprises diluting the sample prior to the partitioning step so that a majority of the partitions include zero or one of the targets.

(A10) For the method denoted as any one of (A1) to (A9), the sample includes four or more targets, and the set of universal probes includes four fluorophores (e.g., four universal probes are used, each universal probe having a different one of four fluorophores).

(A11) For the method denoted as any one of (A1) to (A10), the sample includes six or more targets, and the set of universal probes includes six fluorophores (e.g., six universal probes are used, each universal probe having a different one of six fluorophores); or the sample includes seven or more targets, and the set of universal probes includes seven fluorophores (e.g., seven universal probes are used, each universal probe having a different one of seven fluorophores).

(A12) For the method denoted as any one of (A1) to (A11), the tailed primers each comprise 5′-universal tail, anchor, bridge, foot-3′; or in the 5′ to 3′ direction an anchor, a bridge, and a foot, wherein the primer further includes a universal tail sequence that is bound by a universal probe and is on the 5′ end of the primer or is within the bridge.

(A13) For the method denoted as any one of (A1) to (A12), the set of probes includes a first universal probe linked to a fluorophore of a first color and a second universal probe linked to a fluorophore of a second color, and wherein at least three targets are amplified using tailed primers that include: (i) a first forward tailed primer comprising a first tail with a first universal binding sequence linked to a first forward priming sequence, and (ii) a first reverse primer; (iii) a second forward tailed primer comprising the first tail linked to a second forward priming sequence, and (iv) a second reverse primer; (v) a third forward tailed primer comprising a second tail with a second universal binding sequence linked to the second forward priming sequence, and (vi) the second reverse primer; and (vii) a fourth forward tailed primer comprising the second tail linked to a third forward priming sequencing, and (viii) a third reverse primer.

(A14) For the method denoted as any one of (A1) to (A13), the plurality of partitions comprises microwells or droplets.

(A15) For the method denoted as any one of (A1) to (A14), the sample includes, is suspected of including, or is being tested for six or more targets and the set of probes includes six fluorophores, and color is read using a six-channel digital PCR instrument; or the sample includes, is suspected of including, or is being tested for seven or more targets and the set of probes includes seven fluorophores, and color is read using a seven-channel digital PCR instrument.

(A16) For the method denoted as (A15), the reporting step comprises detecting colors from the plurality of partitions two color channels at a time, in three detection operations.

(A17) For the method denoted as any one of (A1) to (A16), the plurality of partitions are droplets and the amplifying step comprises thermocycling the droplets within a reaction tube or well of a plate, and wherein the determining or detecting step comprises flowing the droplets one-at-a-time past an excitation source and a detector.

(A18) For the method denoted as any one of (A1) to (A17), the method further comprises reading the sample for at least seven targets using at least six colors by detecting two of the six colors, in two channels at a time.

(A19) For the method denoted as any one of (A1) to (A18), the reporting step comprises plotting an amount of a first color and a second color detected from the plurality of partitions as points on a graph and identifying clusters of the points on the graph corresponding to the presence of any of the targets.

(A20) For the method denoted as any one of (A1) to (A19), the amplifying step is performed with primers that comprise DNA oligonucleotides, and the set of probes comprises DNA oligonucleotides linked to fluorophores.

(A21) For the method denoted as any one of (A1) to (A20), the number of the plurality of partitions in which one or more probes bound to a corresponding amplicon is determined by radial multiplexing or intensity multiplexing, or a combination of radial multiplexing and intensity multiplexing

(A22) For the method denoted as any one of (A1) to (A21), the universal probes comprise DNA oligonucleotides linked to quenchers.

(A23) For the method denoted as any one of (A1) to (A22), the amplifying step is performed with a high-fidelity DNA polymerase optionally lacking an exonuclease activity.

(A24) For the method denoted as (A1) to (A23), the set of universal probes comprises a number of universal probes greater than the number of tail sequences of the tailed amplicons or tailed primers.

(A25) For the method denoted as any one of (A1) to (A24), one of the plurality of targets is amplified with more than one tailed primer to yield at least a first tailed amplicon and a second tailed amplicon.

(A26) For the method denoted as (A25), the first tailed amplicon and the second tailed amplicon anneal to a first universal probe and a second universal probe, respectively, the universal probes labeled with different colors.

(A27) For the method denoted as (A25), one of the plurality of targets is amplified with at least 3, at least 4, at least 5, or at least 6 different tailed primers.

(A28) For the method denoted as any one of (A1) to (A27), at least one of the plurality of targets is amplified with a mixture of tailed and untailed forward primers, wherein the priming portion of the forward untailed primer is identical to the priming portion of the forward tailed primer or is configured to amplify from the same target nucleic acid starting material; and/or at least one of the plurality of targets is amplified with a mixture of tailed and untailed reverse primers, wherein the priming portion of the reverse untailed primer is identical to the priming portion of the reverse tailed primer or is configured to amplify from the same target nucleic acid starting material.

(A29) For the method denoted as (A28), at least one of the plurality of targets is amplified with a mixture of tailed and untailed forward primers and a mixture of tailed and untailed reverse primers.

(A30) For the method denoted as (A29), the tailed forward primer corresponds to at least a first universal probe(s) and tailed reverse primer corresponds to the same universal probe(s).

(A31) For the method denoted as (A29), the tailed forward primer corresponds to at least a first universal probe(s) and tailed reverse primer corresponds to different universal probe(s) than the at least a first universal probe(s).

(A32) For the method denoted as (A31), the determining a number of the plurality of partitions in which the respective sets of universal probes bound to the amplicons by detecting light of the respective color from the plurality of partitions requires that the respective colors from probes corresponding to both the forward and reverse tailed primers be present in the same partition.

(A33) For the method denoted as any one of (A28) to (A32), the priming portion of the corresponding tailed and untailed primers are not identical but are designed to amplify from the same nucleic acid starting material.

(A34) For the method denoted as any one of (A28) to (A33), wherein the ratio of tailed:untailed primers is in a range of about 3:1 to about 1:3.

(A35) For the method denoted as (A34), the ratio is about 1:1.

(A36) For the method denoted as (A1), the detection method is performed using qPCR rather than digital PCR, optionally wherein the method is performed in bulk without partitioning. The presence or absence and/or quantity of each of the targets is reported in the sample based on which the universal probes bound to their corresponding amplicons.

1. A target detection method comprising:
partitioning a sample comprising a plurality of targets into a plurality of partitions;
amplifying the targets with mixtures of primers in which a plurality are tailed primers to yield amplicons with tails;
annealing universal probes to tails of the amplicons, wherein each of the universal probes is labeled with a respective color;
determining a number of the plurality of partitions in which each universal probe bound to the amplicons by detecting light of the respective color from the plurality of partitions; and
reporting presence or absence of each of the targets in the sample based on the number of the plurality of partitions in which the universal probes bound to the amplicons.

2-3. (canceled)

4. The method of claim 1, wherein each target is amplified using primers with universal tails, each primer provided at a relative concentration specific to each target.

5. The method of claim 1, wherein a set of universal probes at fixed concentrations for each probe is used in combination with a set of tailed primers at a relative concentration specific to the target to be detected.

6. The method of claim 1, wherein for each target, the target is amplified using (i) a mix of forward primers each having a universal tail and provided at a relative concentration specific to that target, and (ii) a reverse primer.

7-9. (canceled)

10. The method of claim 1, wherein the sample includes more than four targets, and the set of universal probes includes four fluorophores.

11. The method of claim 1, wherein the sample includes more than six targets, and the set of probes includes six fluorophores.

12. (canceled)

13. The method of claim 1, wherein the set of probes includes a first universal probe sequence linked to a fluorophore of a first color and a second universal probe sequence linked to a fluorophore of a second color, and wherein at least three targets are amplified using tailed primers that include:
(i) a first forward tailed primer comprising a first tail with a first universal binding sequence linked to a first forward priming sequence, and (ii) a first reverse primer;
(iii) a second forward tailed primer comprising the first tail linked to a second forward priming sequence, and (iv) a second reverse primer;
(v) a third forward tailed primer comprising a second tail with a second universal binding sequence linked to the second forward priming sequence, and (vi) the second reverse primer; and
(vii) a fourth forward tailed primer comprising the second tail linked to a third forward priming sequencing, and (viii) a third reverse primer.

14-17. (canceled)

18. The method of claim 1, further comprising reading the sample for at least seven targets using at least six colors by detecting two of the six colors, in two channels at a time.

19-23. (canceled)

24. The method of claim 5, wherein the set of universal probes comprises a number of probe sequences greater than the number of tail sequences of the tailed amplicons.

25. The method of claim 1, wherein one of the plurality of targets is amplified with more than one tailed primer to yield at least a first tailed amplicon and a second tailed amplicon.

26. The method of claim 25, wherein the first tailed amplicon and the second tailed amplicon anneal to a first universal probe and a second universal probe, respectively, the universal probes labeled with different colors.

27. The method of claim 25, wherein one of the plurality of targets is amplified with at least 3, at least 4, at least 5, or at least 6 different tailed primers.

28. The method of claim 1, wherein one of the plurality of targets is amplified with a mixture of forward tailed and untailed primers, wherein; a priming portion of the forward untailed primer is identical to the priming portion of the forward tailed primer; or priming portions of the tailed and untailed primers are not identical but are designed to amplify from the same nucleic acid starting material.

29. The method of claim 28, wherein one of the plurality of targets is amplified with a mixture of tailed and untailed forward primers and a mixture of tailed and untailed reverse primers.

30. The method of claim 29, wherein the tailed forward primer corresponds to at least a first universal probe(s) and the tailed reverse primer corresponds to the same universal probe(s); or the tailed forward primer corresponds to at least a first universal probe(s) and the tailed reverse primer corresponds to different universal probe(s) than the at least a first universal probe(s).

31-33. (canceled)

34. The method of claim 28, wherein the ratio of tailed:untailed primers is in a range of about 3:1 to about 1:3.

35. The method of claim 34, wherein the ratio is about 1:1.

36-37. (canceled)

38. The method of claim 1, wherein the sample includes seven or more than seven targets, and the set of probes includes seven fluorophores.

39-41. (canceled)

42. The method of claim 1, wherein one of the plurality of targets is amplified with a mixture of reverse tailed and untailed primers, wherein; the priming portion of the reverse untailed primer is identical to the priming portion of the reverse tailed primer; or the priming portion of the tailed and untailed primers are not identical but are designed to amplify from the same nucleic acid starting material.

43. (canceled)

44. The method of claim 42, wherein the ratio of tailed:untailed primers is in a range of about 3:1 to about 1:3.

45. The method of claim 44, wherein the ratio is about 1:1.
